(19) United States
(12) Patent Application Publication
(10) Pub. No.: US 2010/0036101 A1
(43) Pub. Date: Feb. 11, 2010

(54) ANTIBODIES AGAINST NON FUNCTIONAL P2X7 RECEPTOR

(76) Inventors: Angus Gidley-Baird, North Ryde (AU); Julian Alexander Barden, North Ryde (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/445,258

(22) PCT Filed: Oct. 10, 2007
(86) PCT No.: PCT/AU2007/001541

§ 371 Date: Apr. 10, 2009

(30) Foreign Application Priority Data
Oct. 10, 2006 (AU) 2006905609

(51) Int. Cl. C07K 16/00

(52) U.S. Cl. 530/387.3

(57) ABSTRACT

A recombinant or synthetic antibody or fragment thereof, said antibody or fragment thereof including three complementarity determining regions (CDR1_{L} or H, CDR2_{L} or H and CDR3_{L or H}) for forming an antigen binding site that is capable of binding to a non functional P2X7 receptor but not capable of binding to a functional P2X7 receptor.

### FIELD OF THE INVENTION

The present invention relates to the production of monoclonal antibodies from hybridoma cell lines and to synthetic or recombinant antibodies.

### BACKGROUND OF THE INVENTION

Purinergic (P2X) receptors are ATP-gated cation-selective channels. Each receptor is made up of three protein subunits or monomers. To date seven separate genes encoding P2X monomers have been identified: P2X1, P2X2, P2X3, P2X4, P2X5, P2X6, P2X7.

P2X7 receptors are of particular interest as the expression of these receptors is understood to be limited to cells having potential to undergo programmed cell death, such as thymocytes, dendritic cells, lymphocytes, macrophages and monocytes. There is some expression of P2X7 receptors in normal homeostasis, such as on erythrocytes.

Interestingly, a P2X7 receptor containing one or more monomers having a cis isomerisation at Pro210 (according SEQ ID NO: 58 (FIG. 11)) and which is devoid of ATP binding function has been found on cells that are understood to be unable to undergo programmed cell death, such as preneoplastic cells and neoplastic cells. This isoform of the receptor has been referred to as a “non functional” receptor.

Antibodies generated from immunisation with a peptide including Pro210 in cis bind to non functional P2X7 receptors. However, they do not bind to P2X7 receptors capable of binding ATP. Accordingly, these antibodies are useful for selectively detecting many forms of carcinoma and haemopoietic cancers and to treatment of some of these conditions.

WO02/057306A1 and WO03/020762A1 both discuss a probe for distinguishing between functional P2X7 receptors and non functional P2X7 receptors in the form of a monoclonal antibody.

Affinity matured antibodies are useful in therapeutic and diagnostic applications that require antibodies having a relatively high affinity for a given target or biomarker. Candidates for affinity maturation are generally antibodies that have been subjected to numerous in vitro and in vivo studies to determine binding affinity, tissue and cellular specificity, clearance and other characteristics that are relevant to diagnostic and therapeutic applications. These studies require a plentiful supply of antibody in the form of an ascites produced by a hybridoma.

To date it has been very difficult to obtain a hybridoma that generates useful amounts of antiserum against non functional P2X7 receptors. Indeed, apart from the hybridomas and antibodies forming part of this invention, the Applicant is unaware of any other hybridomas or monoclonal antibodies against anti-non functional P2X7 receptors. This has substantially prevented the in vitro affinity maturation of antibodies against the non functional P2X7 receptor.

There remains a need for monoclonal antibodies that bind to non functional P2X7 receptors, but not to functional P2X7 receptors, especially high affinity antibodies.

### SUMMARY OF THE INVENTION

In a first aspect there is provided a recombinant or synthetic antibody or fragment thereof, said antibody or fragment thereof including three complementarity determining regions (CDR1_{L}, CDR2_{L} and CDR3_{L}) for forming an antigen binding site that is capable of binding to a non functional P2X7 receptor but not capable of binding to a functional P2X7 receptor, wherein the CDR1_{L} includes a peptide having a sequence defined by the following formula:

AA_{1L} AA_{2L} AA_{3L} AA_{4L} AA_{5L} AA_{6L} AA_{7L} AA_{8L} AA_{9L} AA_{10L} AA_{11L} AA_{12L} AA_{13L} AA_{14L} AA_{15L} AA_{16L} AA_{17L}

wherein
AA_{1L} is a basic amino acid residue;
AA_{2L} is a small, nucleophilic, hydrophilic or hydrophobic amino acid residue;
AA_{3L} is a nucleophilic or hydrophilic amino acid residue;
AA_{4L} is a basic, amide, nucleophilic, hydrophilic or acidic amino acid residue;
AA_{5L} is a nucleophilic, hydrophilic or small amino acid residue;
AA_{6L} is a hydrophobic or small amino acid residue;
AA_{7L} is a nucleophilic, hydrophilic or hydrophobic amino acid residue or absent;
AA_{8L} is a nucleophilic, aromatic or basic amino acid residue or absent;
AA_{9L} is a hydrophobic, nucleophilic or hydrophilic amino acid residue or absent;
AA_{10L} is a nucleophilic or hydrophilic amino acid residue or absent;
AA_{11L} is a nucleophilic or hydrophilic amino acid residue or absent;
AA_{12L} is a small or hydrophilic amino acid residue or absent;
AA_{13L} is a aromatic hydrophobic, acidic or hydrophilic amino acid residue or absent;
AA_{14L} is a nucleophilic or hydrophilic amino acid residue;
AA_{15L} is a aromatic or hydrophobic amino acid residue;
AA_{16L} is a hydrophobic or small amino acid residue; and
AA_{17L} is a nucleophilic, hydrophilic, amide, acidic aromatic, hydrophobic, small or basic amino acid residue.

In a second aspect there is provided a recombinant or synthetic antibody or fragment thereof, said antibody or fragment thereof including three complementarity determining regions (CDR1_{L}, CDR2_{L} and CDR3_{L}) for forming an antigen binding site that is capable of binding to a non functional P2X7 receptor but not capable of binding to a functional P2X7 receptor, wherein the CDR2_{L} includes a peptide having a sequence defined by the following formula:

AA_{18L} AA_{19L} AA_{20L} AA_{21L} AA_{22L} AA_{23L} AA_{24L}

wherein
AA_{18L} is hydrophobic, basic, small, hydrophilic, or acidic amino acid residue;
AA_{19L} is small, hydrophobic, nucleophilic, or hydrophilic amino acid residue;
AA_{20L} is nucleophilic, hydrophilic or acidic amino acid residue;
AA_{21L} is hydrophilic, aromatic, hydrophobic or nucleophilic amino acid residue;
AA_{22L} is hydrophobic or basic amino acid residue;
AA_{23L} is acidic, aromatic, hydrophobic or small amino acid residue; and
AA_{24L} is nucleophilic, hydrophilic hydrophobic or acidic amino acid residue.

In a third aspect there is provided a recombinant or synthetic antibody or fragment thereof, said antibody or fragment thereof including three complementarity determining regions (CDR1_{L}, CDR2_{L} and CDR3_{L}) for forming an antigen binding site that is capable of binding to a non functional P2X7 receptor but not capable of binding to a functional P2X7 receptor, wherein the CDR3_{L} includes a peptide having a sequence defined by the following formula:

AA_{25L}AA_{26L}AA_{27L}AA_{28L}AA_{29L}AA_{30L}AA_{31L}AA_{32L}AA_{33L}

wherein:
AA_{25L} is a amide, nucleophilic, hydrophilic, aromatic, hydrophobic or small amino acid residue;
AA_{26L} is a nucleophilic, aromatic, basic, amide or hydrophobic amino acid residue;
AA_{27L} is a nucleophilic, hydrophilic, hydrophobic, small, aromatic or basic amino acid residue;
AA_{28L} is a basic, nucleophilic, hydrophilic, hydrophobic or aromatic amino acid residue;
AA_{29L} is a acidic, aromatic, hydrophobic, nucleophilic or hydrophilic amino acid residue;
AA_{30L} is a hydrophobic, aromatic, hydrophilic or nucleophilic amino acid residue;
AA_{31L} is a hydrophobic, nucleophilic, aromatic or basic amino acid residue;
AA_{32L} is a basic, aromatic or hydrophobic amino acid residue; and
AA_{33L} is a nucleophilic, hydrophilic or hydrophobic amino acid residue.

In a fourth aspect there is provided a recombinant or synthetic antibody or fragment thereof, said antibody or fragment thereof including three complementarity determining regions (CDR1_{H}, CDR2_{H and CDR}3_{H}) for forming an antigen binding site that is capable of binding to a non functional P2X7 receptor but not capable of binding to a functional P2X7 receptor, wherein the CDR1_{H} includes a peptide having a sequence defined by the following formula:

AA_{1H} AA_{2H} AA_{3H} AA_{4H} AA_{5H}

wherein
AA_{1H} is a nucleophilic, hydrophilic, acidic, small, aromatic or hydrophobic amino acid residue;
AA_{2H} is a small, hydrophilic, aromatic or hydrophobic amino acid residue;
AA_{3H} is a aromatic, hydrophobic, small or hydrophilic amino acid residue;
AA_{4H} is a aromatic or hydrophobic amino acid residue; and
AA_{5H} is a hydrophilic, nucleophilic, aromatic or basic amino acid residue.

In a fifth aspect there is provided a recombinant or synthetic antibody or fragment thereof, said antibody or fragment thereof including three complementarity determining regions (CDR1_{H}, CDR2_{H} and CDR3_{H}) for forming an antigen binding site that is capable of binding to a non functional P2X7 receptor but not capable of binding to a functional P2X7 receptor, wherein the CDR2_{H} includes a peptide having a sequence defined by the following formula:

wherein

AA_{6H} AA_{7H} AA_{8H} AA_{9H} AA_{10H} AA_{11H} AA_{12H} AA_{13H} AA_{14H} AA_{15H} AA_{6H} AA_{17H} AA_{18H} AA_{19H} AA_{20H} AA_{21H} AA_{22H} AA_{23H} AA_{24H}
AA_{6H} is a aromatic, hydrophobic, small, hydrophilic, nucleophilic or acidic amino acid residue;
AA_{7H} is a hydrophobic amino acid residue;
AA_{8H} is a hydrophilic, acidic, small or basic amino acid residue;
AA_{9H} is a hydrophobic, nucleophilic or hydrophilic amino acid residue or absent;
AA_{10H} is a basic amino acid residue or absent;
AA_{11H} is a nucleophilic or hydrophilic amino acid residue or absent;
AA_{12H} is a aromatic, hydrophobic, hydrophilic, small or acidic amino acid residue;
AA_{13H} is a nucleophilic, hydrophobic or small amino acid residue;
AA_{14H} is a small, hydrophilic, aromatic or hydrophobic amino acid residue;
AA_{15H} is a hydrophilic, nucleophilic, small, basic, acidic or amide amino acid residue;
AA_{16H} is a nucleophilic, hydrophilic, small or hydrophobic amino acid residue;
AA_{17H} is a aromatic, hydrophobic, nucleophilic, hydrophilic or basic amino acid residue;
AA_{18H} is a aromatic or hydrophobic amino acid residue;
AA_{19H} is a hydrophilic, hydrophobic, small or nucleophilic amino acid residue;
AA_{20H} is a hydrophobic, amide or acidic amino acid residue;
AA_{21H} is a nucleophilic, hydrophilic, basic or acidic amino acid residue;
AA_{22H} is a hydrophobic or aromatic amino acid residue;
AA_{23H} is a hydrophilic or basic amino acid residue; and
AA_{24H} is a nucleophilic, hydrophilic or small amino acid residue.

In a sixth aspect there is provided a recombinant or synthetic antibody or fragment thereof, said antibody or fragment thereof including three complementarity determining regions (CDR1_{H}, CDR2_{H} and CDR3_{H}) for forming an antigen binding site that is capable of binding to a non functional P2X7 receptor but not capable of binding to a functional P2X7 receptor, wherein the CDR3_{H} includes a peptide having a sequence defined by the following formula:

AA_{25H} AA_{26H} AA_{27H} AA_{28H} AA_{29H} AA_{30H} AA_{31H} AA_{32H} AA_{33H} AA_{34H} AA_{35H} AA_{36H} AA_{37H}

wherein
AA_{25H} is a small, hydrophilic or nucleophilic amino acid residue or absent;
AA_{26H} is a basic or hydrophobic amino acid residue or absent;
AA_{27H} is a small hydrophilic, hydrophobic or aromatic amino acid residue or absent;
AA_{28H} is a hydrophobic, nucleophilic, hydrophilic, acidic or aromatic amino acid residue or absent;
AA_{29H} is a aromatic, hydrophobic, small, hydrophilic, nucleophilic or acidic amino acid residue;
AA_{30H} is a aromatic, hydrophobic, nucleophilic, hydrophilic or small amino acid residue or absent;
AA_{31H} is a aromatic, hydrophobic, small, nucleophilic or hydrophilic amino acid residue or absent;
AA_{32H} is a basic, hydrophobic, nucleophilic, hydrophilic or aromatic amino acid residue or absent;
AA_{33H} is a nucleophilic, hydrophilic, or acidic amino acid residue or absent;
AA_{34H} is a small, hydrophilic, aromatic or hydrophobic amino acid residue or absent;
AA_{35H} is a aromatic or hydrophobic amino acid residue or absent;
AA_{36H} is a small, hydrophobic, acidic, nucleophilic or hydrophilic amino acid residue; and
AA_{37H} is a aromatic, hydrophobic, nucleophilic or hydrophilic amino acid residue.

In another aspect there is provided a use of a peptide having a sequence defined by a formula described above for forming an antigen binding site that is capable of binding to a non functional P2X7 receptor but not capable of binding to a functional P2X7 receptor.

In another aspect there is provided an immune complex formed from the binding of an antibody or fragment thereof described above to a non functional P2X7 receptor, monomer or fragment thereof, or to a peptide shown in SEQ ID NO: 58 from position 200 to 216.

In another aspect there is provided a method for determining whether a cell, tissue or extra cellular body fluid includes a non functional P2X7 receptor, monomer or fragment thereof including:
contacting a cell, tissue or extra cellular body fluid with an antibody or fragment thereof described above in conditions for forming an immune complex as described above, and
detecting whether an immune complex has been formed,
   wherein detection of an immune complex determines that a cell, tissue or extra-cellular body fluid includes a non functional P2X7 receptor, monomer or fragment thereof.

In another aspect there is provided a kit or composition for determining whether a cell, tissue or extra-cellular body fluid contains a non functional P2X7 receptor, monomer or fragment thereof including:
an antibody or fragment thereof described above for forming an immune complex with a non functional P2X7 receptor, monomer or fragment thereof and optionally;
a further antibody for binding to the antibody or fragment thereof or the non functional P2X7 receptor, monomer or fragment thereof;
written instructions for use of the kit in a method described above.

In other embodiments there is provided a pharmaceutical composition including an antibody or fragment thereof as described above together with a pharmaceutically acceptable carrier, diluent or excipient.

In related embodiments there is provided a method of treatment of a disease characterised by the expression of a non ATP-binding P2X7 receptor, monomer or fragment thereof including the step of providing an antibody or fragment thereof as described above, or a peptide as described above to an individual requiring said treatment.

### BRIEF DESCRIPTION OF THE FIGURES

FIG. 1 shows the ClustalW protein sequence alignment of the 10 light chain variable regions comprising the most preferred light chain CDRs (CDR_{L}s) of the invention.

FIG. 2 shows the ClustalW protein sequence alignment of the 9 heavy chain variable regions comprising the most preferred heavy chain CDR_{S} (CDR_{H}s) of the invention.

FIG. 3 shows the number and frequency of occurrence of amino acids at each position within the CDR1_{L}, CDR2_{L} and CDR3_{L} regions for the murine germline light chain sequences.

FIG. 4 shows the number and frequency of occurrence of amino acids at each position within the CDR1_{H} and CDR2_{H} regions for the murine germline heavy chain sequences.

FIG. 5 shows an alignment of the CDR1_{L} sequences of invention. The values indicate the frequency of occurrence of the amino acid in the murine germline light chain CDR1_{L} sequences at that position.

FIG. 6 shows an alignment of the CDR2_{L} sequences of invention. The values indicate the frequency of occurrence of the amino acid in the murine germline light chain CDR2_{L} sequences at that position.

FIG. 7 shows an alignment of the CDR3_{L} sequences of invention. The values indicate the frequency of occurrence of the amino acid in the murine germline light chain CDR3_{L} sequences at that position.

FIG. 8 shows an alignment of the CDR1_{H} sequences of invention. The values indicate the frequency of occurrence of the amino acid in the murine germline heavy chain CDR1_{H} sequences at that position.

FIG. 9 shows an alignment of the CDR2_{H} sequences of invention. The values indicate the frequency of occurrence of the amino acid in the murine germline heavy chain CDR2_{H} sequences at that position.

FIG. 10 shows the alignment of the CDR3_{H} sequences of the invention.

FIG. 11 shows the sequence of the human P2X7 receptor.

FIGS. 12 to 17 show the sequences of various slice variants of the human P2X7 receptor.

### DETAILED DESCRIPTION

The anti P2X7 antisera against non functional P2X7 receptors available at the time of the invention have all been polyclonal. Apart from the Applicant's own work, no anti-non functional P2X7 receptor monoclonal antibodies have been made.

The inventors have attempted to obtain anti non functional P2X7 receptor monoclonal antibodies using techniques for monoclonal antibody production known in the art. In accordance with conventional techniques, a key step in this process has been to screen and to select for hybridomas for antibody production that produce supernatants having high affinity for the peptide immunogen against which they have been raised (Goding, J. W. Monoclonal antibodies: principles and practice: production and application of monoclonal antibodies in cell biology, biochemistry and immunology.—2nd ed. 1986 Academic Press, Harcour Brace Jovnovich, Publishers.)

In forming this invention, the inventors found that these techniques tend to result in hybridomas that exhibit poor growth in mice and that are sensitive to cell culture techniques including passaging, freezing, thawing and seeding. Consequently the inventors found it to be very difficult to generate amounts of antibodies from hybridomas selected according to the conventional monoclonal antibody techniques that are sufficient to complete the in vitro and in vivo studies required to identify candidates for affinity maturation.

Further, the inventors found that the hybridomas selected for antibody production on the basis of high affinity binding to peptide immunogen tend to produce antibodies that also have high affinity for non functional P2X7 receptors expressed on the surface of live cells. The inventors now believe that these antibodies bind to the hybridomas that produce them with high affinity.

This has been a surprising finding given that the peptide immunogen is based on a human P2X7 receptor, whereas the hybridoma is formed from non human cells.

Still further, in forming this invention the inventors have screened for hybridomas that secrete antibodies that bind to non functional P2X7 receptors expressed on live cells with low affinity, and then determined the capacity of these hybridoma cells to grow in mice and tissue culture. The inventors have surprisingly found that the hybridomas showed a much better potential for growth and stability than those that secrete antibodies that bind to receptors on live cells with high affinity. As a result, the inventors have been able to produce large amounts of monoclonal antibody to non functional P2X7 receptors and have been able to complete studies for identifying candidates for affinity maturation. Surprisingly the inventors have been able to affinity mature these low affinity antibodies 100 fold without losing the specificity of binding for non functional receptors.

Accordingly, in one embodiment of the first aspect of the invention there is provided a recombinant or synthetic antibody or fragment thereof, said antibody or fragment thereof including three complementarity determining regions (CDR1_{L}, CDR2_{L} and CDR3_{L}) for forming an antigen binding site that is capable of binding to a non functional P2X7 receptor but not capable of binding to a functional P2X7 receptor, wherein the CDR1_{L} includes a peptide having a sequence defined by the following formula:

AA_{1L} AA_{2L} AA_{3L} AA_{4L} AA_{5L} AA_{6L} AA_{7L} AA_{8L} AA_{9L} AA_{10L} AA_{11L} AA_{12L} AA_{13L} AA_{14L} AA_{15L} AA_{16L} AA_{17L}

wherein
AA_{1L} is a basic amino acid residue;
AA_{2L} is a small, hydrophilic or hydrophobic amino acid residue;
AA_{3L} is a hydrophilic amino acid residue;
AA_{4L} is a basic, amide, hydrophilic or acidic amino acid residue;
AA_{5L} is a hydrophilic amino acid residue;
AA_{6L} is a hydrophobic or small amino acid residue;
AA_{7L} is a hydrophilic or hydrophobic amino acid residue or absent;
AA_{8L} is a nucleophilic, aromatic or basic amino acid residue or absent;
AA_{9L} is a hydrophobic or hydrophilic amino acid residue or absent;
AA_{10L} is a hydrophilic amino acid residue or absent;
AA_{11L} is a hydrophilic amino acid residue or absent;
AA_{12L} is a hydrophilic or small amino acid residue or absent;
AA_{13L} is a aromatic, acidic or hydrophilic amino acid residue or absent;
AA_{14L} is a hydrophilic amino acid residue;
AA_{15L} is a aromatic amino acid residue;
AA_{16L} is a hydrophilic or a small amino acid residue; and
AA_{17L} is a nucleophilic, amide, acidic, aromatic, hydrophilic, small or a basic amino acid residue.

Antibodies are immunoglobulin molecules comprised of four polypeptide chains, two heavy (H) chains and two light (L) chains, interconnected by disulfide bonds. Each polypeptide chain comprises a constant region (C) and a variable region (V). Each polypeptide chain is also organised into a series of domains. The light chain polypeptides comprise two domains, one corresponding to the C region and the other to the V region. The heavy chain polypeptides comprise four domains, one corresponding to V region and three domains (C_{H}1, C_{H}2 and C_{H}3) corresponding to the constant region. The variable regions for both the heavy (V_{H}) and light (V_{L}) chains further comprise three hypervariability regions termed complementarity determining regions (CDRs) separated by four framework regions. A “framework region” (FR) is an amino acid sequence that forms a framework for the hypervariability sequences that comprise each of the CDRs. The CDRs and FRs are arranged from amino terminus to carboxy terminus in the order: FR1, CDR1, FR2, CDR2, FR3, CDR3, FR4. Framework regions are generally well known in the art. A framework region is generally between about 10 to 50 amino acids residues in length. In certain embodiments, framework regions are either 15, 19, 32, 42 or 43 amino acids in length.

Fragment of antibodies generally include a combination of CDRs that form an antigen binding site that is capable of binding to a non functional P2X7 receptor, but not capable of binding to a functional P2X7 receptor. Examples include but are not limited to, dAb, Fab, Fd, Fv, CDRs, F(ab′)₂, and scFv molecules, and include diabodies and multibodies. A dAb molecule comprises a V_{H} or V_{L} domain; a Fab molecule comprises the V_{L}, V_{H}, C_{L} and C_{H}1 domains; a Fd molecule comprises the V_{H} and C_{H}1 domains; F(ab′)₂ is a bivalent molecule comprising two Fab fragments linked by a disulfide bridge at the hinge region; and a F_{V} molecule comprises the V_{L} and V_{H} domains located on a single polypeptide. Although the V_{L} and V_{H} domains of the F_{V} molecule are coded for by separate genes, it is possible to use a synthetic linker that enables these genes to encode a single polypeptide chain in which the V_{L} and V_{H} regions pair to form monovalent molecules. These molecules are known as single chain F_{V} (scFv).

The antibodies or fragments thereof may be “diabodies” and “multibodies”. These may be bivalent/bispecific and trivalent/trispecific molecules, respectively, in which the V_{H} and V_{L} domains are expressed on a single polypeptide chain, but using a linker that is too short to allow for pairing between the two domains on the same chain, thereby forcing the domains to pair with complementary domains of other chains and creating two or more antigen binding sites. Antibodies or fragments thereof that are bi- or multi-specific bind more than one type of epitope when all antigen binding sites on the antibody or fragment are bound to antigen. For example, a bispecific antibody may have an antigen binding site for epitope A and an antigen binding site for epitope B. Further, epitope A and epitope B may be located on the same or different molecules.

A recombinant antibody or fragment thereof is generally an antibody or fragment that has been produced by recombinant DNA technology, examples of which are discussed below. A synthetic antibody or fragment thereof is generally an antibody or fragment that has been produced by peptide synthesis technology.

An antigen binding site is generally a region of an antibody that binds to an antigenic determinant or epitope. An antigen binding site is generally formed from an assembly of CDRs in the context of the FRs discussed above. These regions may be located on separate polypeptide chains, so that, for example, the antigen binding site may be formed from a heavy and light chain, or fragments of these chains having these regions. Alternatively, these regions may be located on a light or heavy chain only, so that the antigen binding site is formed from a light or heavy chain only or fragments of these chains having these regions.

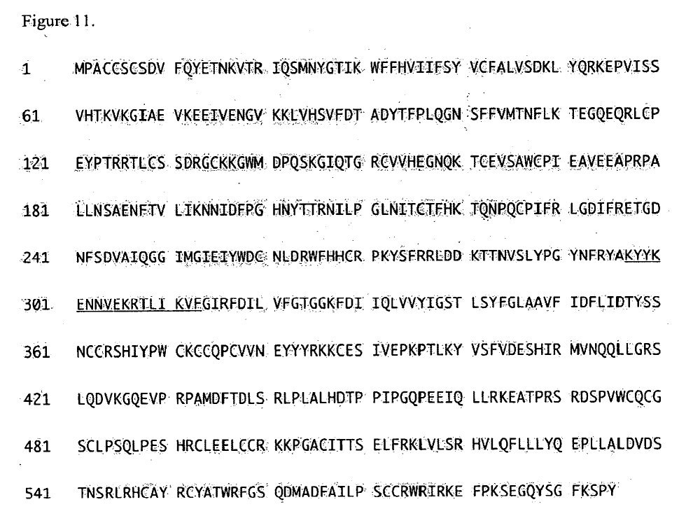

A non functional P2X7 receptor generally contains one or more monomers having a cis isomerisation at Pro210 (according to SEQ ID NO: 58 (FIG. 11)). The receptor is non functional or abnormal in the sense that, in contrast to a normal P2X7 receptor, it is devoid of ATP binding function. Non functional P2X7 receptors have been found on cells that are understood to be unable to undergo programmed cell death, such as preneoplastic cells and neoplastic cells.

The amino acid residues described in this specification as hydrophilic, nucleophilic, hydrophobic, acidic, basic, aromatic, amide, small or disulphide are identified in Table 1. For example, the amino acids valine, leucine and isoleucine are hydrophobic, while serine is hydrophilic. Tyrosine which has an aromatic side chain is hydrophobic, while histidine which also has an aromatic side chain is basic.

TABLE I

| | Biochemical Properties |
|---|---|
| Amino Acid | of Side Chain |
| Alanine (A) | small |
| Cysteine(C) | disulphide |
| Aspartate (D) | acidic |
| Glutamate(E) | acidic |
| Phenylalanine (F) | aromatic, hydrophobic |
| Glycine (G) | small, hydrophilic |
| Histidine (H) | nucleophilic, aromatic, basic |
| Isoleucine (I) | hydrophobic |
| Lysine (K) | basic |
| Leucine (L) | hydrophobic |
| Methionine (M) | hydrophobic |
| Asparagine (N) | hydrophilic |
| Proline (P) | hydrophobic |
| Glutamine (Q) | amide |
| Arginine (R) | basic |
| Serine (S) | nucleophilic, hydrophilic |
| Threonine (T) | nucleophilic, hydrophilic |
| Valine (V) | hydrophobic |
| Tryptophan (W) | aromatic, hydrophobic |
| Tyrosine (Y) | aromatic, hydrophobic |

In certain embodiments, the word “absent” with reference to the above described formulae (for example “AA_{13L} is a aromatic, acidic or hydrophilic amino acid residue or absent”) means that the residue at the given position may not be one as described (in the example, at AA_{13L} it may not be aromatic, acidic or hydrophilic). In the circumstances, the residue at the given position is generally a residue as is defined at the next position of the formula (for example, where AA_{13L} is ‘absent’, the amino acid residue at position 13 is selected from those listed as alternatives at position 14: “AA_{14L} is a hydrophilic amino acid residue”).

In another embodiment of the first aspect of the invention, AA_{1L} is R; AA_{2L} is A, S or V; AA_{3L} is S; AA_{4L} is K, Q, T or E; AA_{5L} is S, N or G; AA_{6L} is V, L, I or A; AA_{7L} is S, V, L or absent; AA_{8L} is H or absent; AA_{9L} is I, S, T or absent; AA_{10L} is T or absent; AA_{11L} is S, N or absent, AA_{12L} is G, A or absent; AA_{13L} is Y, D, N or absent; AA_{14L} is S, T or N; AA_{15L} is Y or F; AA_{16L} is M, L or A; and AA_{17L} is S, H, Q, E, Y, N or A.

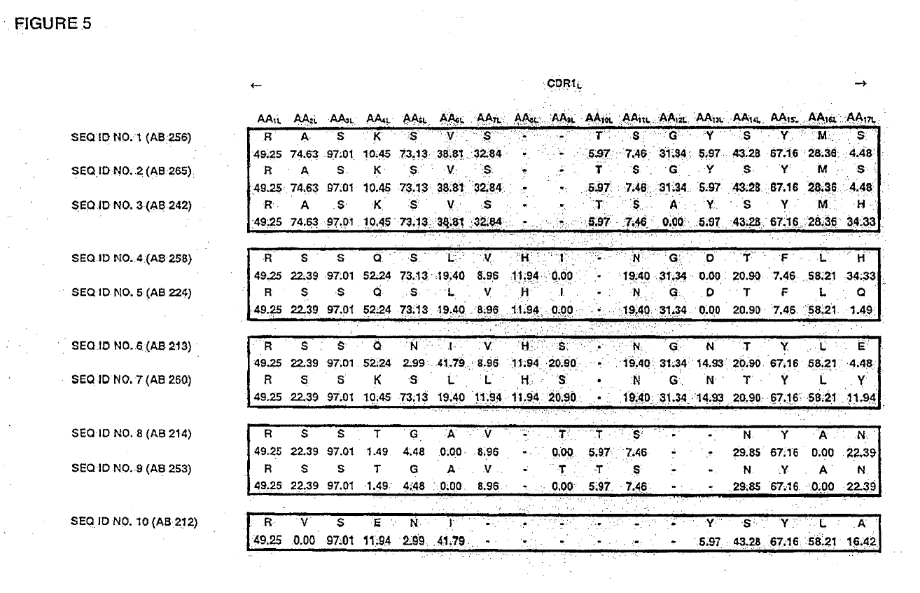

In yet a further embodiment of the first aspect, the peptide forming the CDR1_{L} is selected from the group consisting of SEQ ID NO.1, SEQ ID NO.2, SEQ ID NO.3, SEQ ID NO.4, SEQ ID NO.5, SEQ ID NO.6, SEQ ID NO.7, SEQ ID NO.8, SEQ ID. NO.9, and SEQ ID NO.10 (FIG. 5).

In one embodiment of the second aspect of the invention,
AA_{18L} is a hydrophobic, basic, hydrophilic, or acidic amino acid residue;
AA_{19L} is a small, hydrophobic, or hydrophilic amino acid residue;
AA_{20L} is a hydrophilic or acidic amino acid residue;
AA_{21L} is a hydrophilic or aromatic amino acid residue;
AA_{22L} is a hydrophobic or basic amino acid residue;
AA_{23L} is a acidic, aromatic or small amino acid residue; and
AA_{24L} is a hydrophilic, hydrophobic or acidic amino acid residue.

In another embodiment of the second aspect of the invention AA_{18L} is L, K, R, G, D or N; AA_{19L} is A, V, M or T; AA_{20L} is S, N or E; AA_{21L} is N, Y or T; AA_{22L} is L or R; AA_{23L} is E, F or A; and AA_{24L} is S, P or E.

In another embodiment of the second aspect of the invention, the peptide forming the CDR2_{L} is selected from the group consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20.

In an embodiment according to the third aspect of the invention,
AA_{25L} is a amide, hydrophilic, hydrophobic or small amino acid residue;
AA_{26L} is a nucleophilic, aromatic, basic, amide or hydrophobic amino acid residue;
AA_{27L} is a hydrophilic, hydrophobic, nucleophilic, aromatic or basic amino acid residue;
AA_{28L} is a basic, hydrophilic, hydrophobic or aromatic amino acid residue;
AA_{29L} is a acidic, aromatic, hydrophobic, or hydrophilic amino acid residue;
AA_{30L} is a hydrophobic or hydrophilic amino acid residue;
AA_{31L} is a hydrophobic, nucleophilic, aromatic or basic amino acid residue;
AA_{32L} is a basic, aromatic or hydrophobic amino acid residue; and
AA_{33L} is a hydrophilic or hydrophobic amino acid residue.

In yet a further embodiment of the third aspect of the invention AA_{25L} is Q, S, F, M, A or G; AA_{26L} is H, Q or L; AA_{27L} is S, I, G, H, W or R; AA_{28L} is R, T, S, L, Y or N; AA_{29L} is E, D, F, L, S or V; AA_{30L} is L, V, Y, N or S; AA_{31L} is P or H; AA_{32L} is R, W, L or P; and AA_{33L} is T or V.

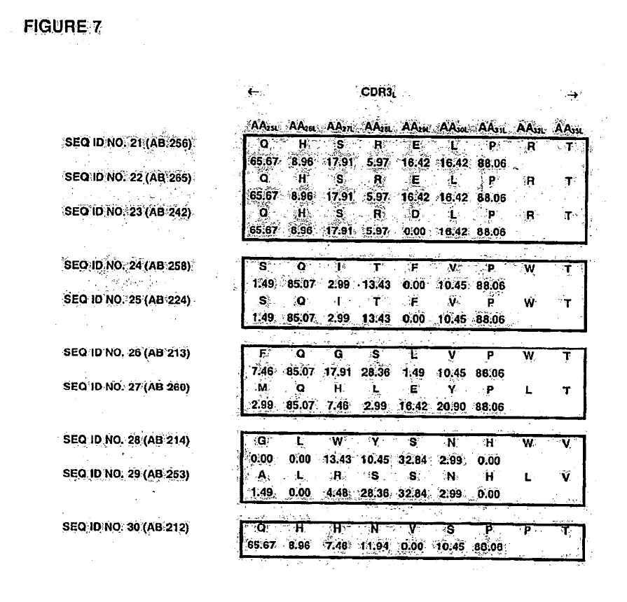

In yet a further embodiment of the third aspect of the invention, the peptide forming CDR3_{L} is selected from group consisting of SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29 and SEQ ID NO. 30 (FIG. 7).

In one embodiment according to the fourth aspect of the invention
AA_{1H} is a hydrophilic, acidic or aromatic amino acid residue;
AA_{2H} is a hydrophilic or aromatic amino acid residue;
AA_{3H} is a aromatic or small amino acid residue;
AA_{4H} is a hydrophobic amino acid residue; and
AA_{5H} is a hydrophilic or nucleophilic amino acid residue.

In an embodiment according to the fourth aspect of the invention AA_{1H} is S, E, G, Y or N; AA_{2H} is G, Y or H; AA_{3H} is Y, A, W or G; AA_{4H} is W, L or M; and AA_{5H} is N, H or S.

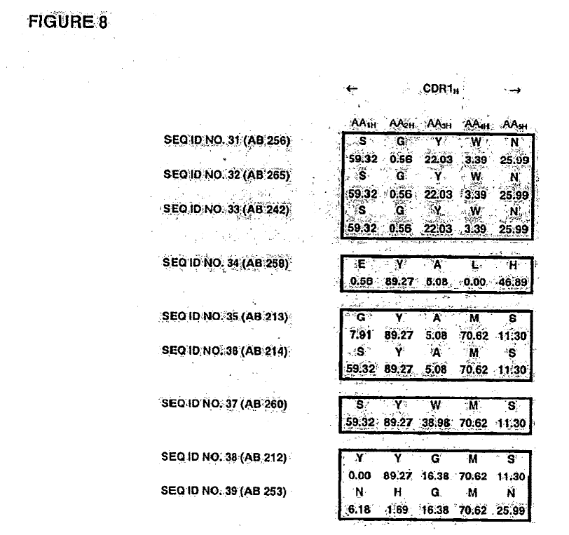

In yet further embodiments according to the fourth aspect of the invention, the peptide forming CDR1_{H} is selected from group consisting of SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38 AND SEQ ID NO. 39 (FIG. 8).

In an embodiment according to the fifth aspect of the invention
AA_{6H} is an aromatic, hydrophilic or acidic amino acid residue;
AA_{7H} is a hydrophobic amino acid residue;
AA_{8H} is a hydrophilic or basic amino acid residue;
AA_{9H} is a hydrophobic or hydrophilic amino acid residue or absent;
AA_{10H} is a basic amino acid residue or absent;
AA_{11H} is a hydrophilic amino acid residue or absent;
AA_{12H} is an aromatic, hydrophilic or acidic amino acid residue;
AA_{13H} is a hydrophilic amino acid residue;
AA_{14H} is a hydrophilic or aromatic amino acid residue;
AA_{15H} is a hydrophilic, basic, small, acidic or amide amino acid residue;
AA_{16H} is a hydrophilic, small or hydrophobic amino acid residue;
AA_{17H} is a aromatic, hydrophobic or hydrophilic amino acid residue;
AA_{18H} is a aromatic amino acid residue;
AA_{19H} is a hydrophilic, hydrophobic or small amino acid residue;
AA_{20H} is a hydrophobic, amide or acidic amino acid residue;
AA_{21H} is a hydrophilic, basic or acidic amino acid residue;
AA_{22H} is a hydrophobic amino acid residue;
AA_{23H} is a hydrophilic or basic amino acid residue; and
AA_{24H} is a hydrophilic amino acid residue.

In yet a further preferred embodiment according to the fifth aspect of the invention AA_{6H} is Y, G, T, S, E or W; AA_{7H} is I; AA_{8H} is N, D, G or R; AA_{9H} is absent, P, S, L or T; AA_{10H} is K or absent; AA_{11H} is S or absent; AA_{12H} is Y, N, G or D; AA_{13H} is S, N, G or T; AA_{14H} is G or Y; AA_{15H} is N, S, T, G, R, A, E or Q; AA_{16H} is T, A, P or S; AA_{17H} is Y, T, H or I; AA_{18H} is Y; AA_{19H} is N, P, A or S; AA_{20H} is P, Q, D or E; AA_{21H} is S, K or D; AA_{22H} is L, F or V; AA_{23H} is N or K; and AA_{24H} is S or G.

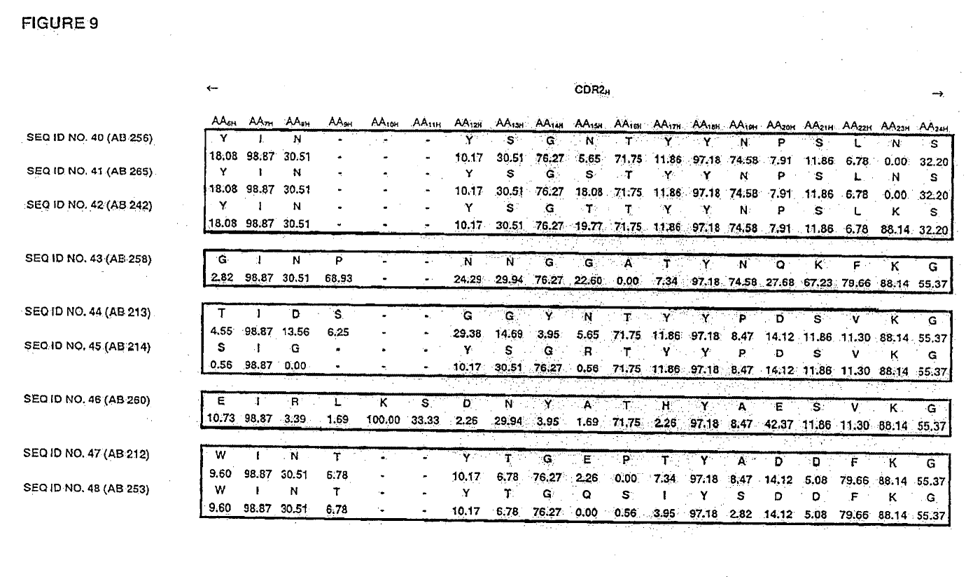

In yet further preferred embodiments according to the fifth aspect of the invention, the peptide forming CDR2_{H} is selected from group consisting of SEQ ID NO. 40, SEQ ID NO. 41, SEQ ID NO. 42, SEQ ID NO. 43, SEQ ID NO 44, SEQ ID NO. 45, SEQ ID NO. 46, SEQ ID NO. 47 and SEQ ID NO. 48 (FIG. 9).

In an embodiment according to the sixth aspect of the invention
AA_{25H} is a small or hydrophilic amino acid residue or absent;
AA_{26H} is a basic or hydrophobic amino acid residue or absent;
AA_{27H} is a small, hydrophilic or hydrophobic amino acid residue or absent;
AA_{28H} is a hydrophobic, hydrophilic or acidic amino acid residue or absent;
AA_{29H} is an aromatic or hydrophilic amino acid residue;
AA_{30H} is a hydrophobic or hydrophilic amino acid residue or absent;
AA_{31H} is a hydrophobic, small or hydrophilic amino acid residue or absent;
AA_{32H} is a basic, hydrophobic or nucleophilic amino acid residue or absent;
AA_{33H} is a hydrophilic, acidic amino acid residue or absent;
AA_{34H} is a small or hydrophobic amino acid residue or absent;
AA_{35H} is a hydrophobic amino acid residue or absent;
AA_{36H} is a small, hydrophobic, acidic or hydrophilic amino acid residue; and
AA_{37H} is a aromatic or nucleophilic amino acid residue.

In yet a further embodiment according to the sixth aspect of the invention AA_{25H}, is G, A, S or absent; AA_{26H}, is R, L, I, K or absent; AA_{27H}, A, N, V, F, Y, G or absent; AA_{28H}, is I, T, E, H, Y, L or absent, AA_{29H}, is Y, F, G or H; AA_{30H}, is Y, F, T, G, V or absent, AA_{31H}, is Y, A, S, N, L or absent, AA_{32H}, is K, M, T, Y or absent; AA_{33H}, is S, D or absent; AA_{34H}, is G, Y, A or absent; AA_{35H}, is F, M or absent; AA_{36H}, is A, V, D or S; AA_{37H}, is Y, F or S.

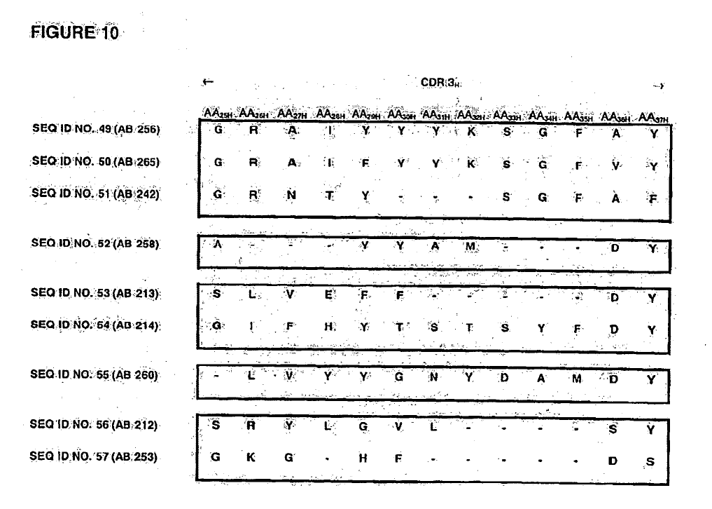

In yet further embodiments according to the sixth aspect of the invention, the peptide forming CDR3_{H} is selected from group consisting of SEQ ID NO. 49, SEQ ID NO. 50, SEQ ID NO. 51, SEQ ID NO. 52, SEQ ID NO. 53, SEQ ID NO. 54, SEQ ID NO. 55 SEQ ID NO. 56 and SEQ ID NO. 57 (FIG. 10).

In certain embodiments, the recombinant or synthetic antibody or fragment thereof may have an affinity for an epitope or antigenic determinant on a non functional P2X7 receptor expressed on a live cell in the range of from 1-10 uM.

The affinity of an antibody for antigen can be quantified by determining an association or disassociation constant. Methods for determining these constants are well known in the art. For example an association constant can be determined by Equilibrium dialysis.

In another aspect there is provided a use of a peptide having a sequence defined by a formula described according to one of the above aspects of the invention for forming an antigen binding site that is capable of binding to a non functional P2X7 receptor but not capable of binding to a functional P2X7 receptor.

The antigen binding site may be comprised within a whole antibody. Alternatively, it may be comprised within a fragment of an antibody, including a fragment as described above.

The peptide for forming the antigen binding site in a antibody or fragment thereof, and indeed the antibody or fragment thereof are generally produced by recombinant technology or peptide synthesis. A recombinant antibody or fragment thereof is generally an antibody or fragment that has been produced by recombinant DNA technology, examples of which are discussed below. A synthetic antibody or fragment thereof is generally an antibody or fragment that has been produced by peptide synthesis technology.

The peptides forming CDRs of the antigen binding site of antibodies or fragments thereof may be prepared using solid-phase synthesis, such as that generally described by Merrifield, J. Am. Chem. Soc., 85: 2149 (1963), although other equivalent chemical syntheses known in the art are employable. Solid-phase synthesis is initiated from the C-terminus of the peptide by coupling a protected α-amino acid to a suitable resin. Such a starting material can be prepared by attaching a α-amino-protected amino acid by an ester linkage to a chloromethylated resin or a hydroxymethyl resin, or by an amide bond to a BHA resin or MBHA resin. The preparation of the hydroxymethyl resin is described by Bodansky et al., Chem. Ind. (London), 38: 1597-1598 (1966). Chloromethylated resins are commercially available from BioRad Laboratories, Richmond, Calif. And from Lab. Systems, Inc. The preparation of such a resin is described by Stewart et al., “Solid Phase Peptide Synthesis” (Freeman & Co., San Francisco 1969), Chapter 1, pp. 1-6. BHA and MBHA resin supports are commercially available and are generally used only when the desired polypeptide being synthesized has an unsubstituted amide at the C-terminus.

The amino acids are coupled to the peptide chain using techniques well known in the art for the formation of peptide bonds. One method involves converting the amino acid to a derivative that will render the carboxyl group more susceptible to reaction with the free N-terminal amino group of the peptide fragment. For example, the amino acid can be converted to a mixed anhydride by reaction of a protected amino acid with ethychloroformate, phenyl chloroformate, sec-butyl chloroformate, isobutyl chloroformate, pivaloyl chloride or like acid chlorides. Alternatively, the amino acid can be converted to an active ester such as a 2,4,5-trichlorophenyl ester, a pentachlorophenyl ester, a pentafluorophenyl ester, a p-nitrophenyl ester, a N-hydroxysuccinimide ester, or an ester formed from 1-hydroxybenzotriazole.

Another coupling method involves use of a suitable coupling agent such as N,N¹-dicyclohexylcarbodiimide or N,N¹-diisopropylcarbodiimide. Other appropriate coupling agents, apparent in those skilled in the art, are disclosed in E Gross & J Meienhofer, The Peptides: Analysis, Structure, Biology, Vol. I: Major Methods of Peptide Bond Formation (Academic Press, New York, 1979).

It should be recognized that the α-amino group of each amino acid employed in the peptide synthesis must be protected during the coupling reaction to prevent side reactions involving their active α-amino function. It should also be recognized that certain amino acids contain reactive side-chain functional groups (eg sulfhydryl, amino, carboxyl, and hydroxyl) and that such functional groups must also be protected with suitable protecting groups to prevent a chemical reaction from occurring at that site during both the initial and subsequent coupling steps. Suitable protecting groups, known in the art, are described in Gross and Meienhofer, The Peptides: Analysis, Structure, Biology, Vol. 3: “Protection of Functional Groups in Peptide Synthesis” (Academic Press, New York 1981).

In the selection of a particular side-chain protecting group to be used in synthesizing the peptides, the following general rules are followed. An α-amino protecting group must render the α-amino function inert under the conditions employed in the coupling reacting, must be readily removable after the coupling reaction under conditions that will not remove side-chain protecting groups and will not alter the structure of the peptide fragment, and must eliminate the possibility of racemization upon activation immediately prior to coupling. A side-chain protecting group must render the side chain functional group inert under the conditions employed in the coupling reaction, must be stable under the conditions employed in removing the α-amino protecting group, and must be readily removable upon completion of the desired amino acid peptide under reaction conditions that will not alter the structure of the peptide chain.

It will be apparent to those skilled in the art that the protecting groups known to be useful for peptide synthesis will vary in reactivity with the agents employed for their removal. For example, certain protecting groups such as triphenylmethyl and 2-(p-biphenylyl)isopropyloxycarbonyl are very labile and can be cleaved under mild acid conditions. Other protecting groups, such as t-butyloxycarbonyl (BOC), t-amyloxycarbonyl, adamantyloxycarbonyl, and p-methoxybenzyloxycarbonyl are less labile and require moderately strong acid, such as trifluoroacetic, hydrochloric, or boron trifluoride in acetic acid, for their removal. Still other protecting groups, such as benzyloxy-carbonyl (CBZ or Z), halobenzyloxycarbonyl, p-nitrobenzyloxycarbonyl cycloalkyloxycarbonyl, and isopropyloxycarbonyl, are even less labile and require stronger acids, such as hydrogen fluoride, hydrogen bromide, or boron trifluoroacetate in trifluoroacetic acid, for their removal. Among the classes of useful amino acid protecting groups are included:

(1) for an α-amino group, (a) aromatic urethane-type protecting groups, such as fluorenylmethyloxycarbonyl (FMOC) CBZ, and substituted CBZ, such as, eg, p-chlorobenzyloxycarbonyl, p-6-nitrobenzyloxycarbonyl, p-bromobenzyloxycarbonyl, and p-methoxybenzyloxycarbonyl, o-chlorobenzyloxycarbonyl, 2,4-dichlorobenzyloxycarbonyl, 2,6-dichlorobenzyloxycarbonyl, and the like; (b) aliphatic urethane-type protecting groups, such as BOC, t-amyloxycarbonyl, isopropyloxycarbonyl, 2-(p-biphenylyl)-isopropyloxycarbonyl, allyloxycarbonyl and the like; (c) cycloalkyl urethane-type protecting groups, such as cyclopentyloxycarbonyl, adamantyloxycarbonyl, and cyclohexyloxycarbonyl; and (d) allyloxycarbonyl. The preferred α-amino protecting groups are BOX or FMOC.

(2) for the side chain amino group present in Lys, protection may be by any of the groups mentioned above in (1) such as BOC, p-chlorobenzyloxycarbonyl, etc.

(3) for the guanidino group of Arg, protection may be by mitro, tosyl, CBZ, adamantyloxycarbonyl, 2,2,5,7,8-pentamethylchroman-6-sulfonyl or 2,3,6-trimethyl-4-methoxyphenylsulfonyl, or BOC.

(4) for the hydroxyl group of Ser, Thr, or Tyr, protection may be, for example, by C1-C4 alkyl, such as t-butyl; benzyl (BAL); substituted BZL, such as p-methoxybenzyl, p-nitrobenzyl, p-chlorobenzyl, o-chlorobenzyl, and 2,6-dichlorobenzyl.

(5) for the carboxyl group of Asp or Glu, protection may be, for example, by esterification using groups such as BZL, t-butyl, cyclohexyl, cyclopentyl, and the like.

(6) for the imidazole nitrogen of His, the tosyl moiety is suitable employed.

(7) for the phenolic hydroxyl group of Tyr, a protecting group such as tetrahydropyranyl, tert-butyl, trityl, BZL, chlorobenzyl, 4-bromobenzyl, or 2,6-dichlorobenzyl is suitably employed. The preferred protecting group is 2,6-dichlorobenzyl.

(8) for the side chain amino group of Asn or Gln, xanthyl (Xan) is preferably employed.

(9) for Met, the amino acid is preferably left unprotected.

(10) for the thio group of Cys, p-methoxybenzyl is typically employed.

The C-terminal amino acid, eg, Lys, is protected at the N-amino position by an appropriately selected protecting group, in the case of Lys, BOC. The BOC-Lys-OH can be first coupled to the benzyhydrylamine or chloromethylated resin according to the procedure set forth in Horiki et al, Chemistry Letters, 165-168 (1978) or using isopropylcarbodiimide at about 25° C. for 2 hours with stirring. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCI in dioxane, and conditions for removal of specific α-amino protecting groups are described in the literature.

After removal of the α-amino protecting group, the remaining α-amino and side-chain protected amino acids are coupled stepwise within the desired order. As an alternative to adding each amino acid separately in the synthesis, some may be coupled to one another prior to addition to the solid-phase synthesizer. The selection of an appropriate coupling reagent is within the skill of the art. Particularly suitable as a coupling reagent is N,N¹-dicyclohexyl carbodiimide or diisopropylcarbodiimide.

Each protected amino acid or amino acid sequence is introduced into the solid-phase reactor in excess, and the coupling is suitably carried out in a medium of dimethylformamide (DMF) or CH₂Cl₂ or mixtures thereof. If incomplete coupling occurs, the coupling procedure is repeated before removal of the N-amino protecting group prior to the coupling of the next amino acid. The success of the coupling reaction at each stage of the synthesis may be monitored. A preferred method of monitoring the synthesis is by the ninhydrin reaction, as described by Kaiser et al., Anal Biochem, 34: 595 (1970). The coupling reactions can be performed automatically using well known methods, for example, a BIOSEARCH 9500™ peptide synthesizer.

Upon completion of the desired peptide sequence, the protected peptide must be cleaved from the resin support, and all protecting groups must be removed. The cleavage reaction and removal of the protecting groups is suitably accomplished simultaneously or stepwise. When the resin support is a chloromethylated polystyrene resin, the bond anchoring the peptide to the resin is an ester linkage formed between the free carboxyl group of the C-terminal residue and one of the many chloromethyl groups present on the resin matrix. It will be appreciated that the anchoring bond can be cleaved by reagents that are known to be capable of breaking an ester linkage and of penetrating the resin matrix.

One especially convenient method is by treatment with liquid anhydrous hydrogen fluoride. This reagent not only will cleave the peptide from the resin but also will remove all protecting groups. Hence, use of this reagent will directly afford the fully deprotected peptide. When the chloromethylated resin is used, hydrogen fluoride treatment results in the formation of the free peptide acids. When the benzhydrylamine resin is used, hydrogen fluoride treatment results directly in the free peptide amines. Reaction with hydrogen fluoride in the presence of anisole and dimethylsulfide at 0° C. for one hour will simultaneously remove the side-chain protecting groups and release the peptide from the resin.

When it is desired to cleave the peptide without removing protecting groups, the protected peptide-resin can undergo methanolysis to yield the protected-peptide-resin can undergo methanolysis to yield the protected peptide in which the C-terminal carboxyl group is methylated. The methyl ester is then hydrolysed under mild alkaline conditions to give the free C-terminal carboxyl group. The protecting groups on the peptide chain then are removed by treatment with a strong acid, such as liquid hydrogen fluoride. A particularly useful technique for methanolysis is that of Moore et al, Peptides, Proc Fifth Amer Pept Symp, M Goodman and J Meienhofer, Eds, (John Wiley, N.Y., 1977), p. 518-521, in which the protected peptide-resin is treated with methanol and potassium cyanide in the presence of crown ether.

Another method of cleaving the protected peptide form the resin when the chloromethylated resin is employed is by ammonolysis or by treatment with hydrazine. If desired, the resulting C-terminal amide or hydrazide can be hydrolysed to the free C-terminal carboxyl moiety, and the protecting groups can be removed conventionally.

It will also be recognized that the protecting group present on the N-terminal α-amino group may be removed preferentially either before or after the protected peptide is cleaved from the support.

If in the peptides being created carbon atoms bonded to four non identical substituents are asymmetric, then the compounds may exist as disastereoisomers, enantiomers or mixtures thereof. The syntheses described above may employ racemates, enantiomers or disastereoisomers as starting materials or intermediates. Disastereomeric products resulting from such syntheses may be separated by chromatographic or crystallization methods. Likewise, enantiomeric product mixtures may be separated using the same techniques or by other methods known in the art. Each of the asymmetric carbon atoms, when present, may be in one of two configurations (R or S) and both are within the scope of the present invention.

Purification of the peptide is typically achieved using conventional procedures such as preparative HPLC (including reversed phase HPLC) or other known chromatographic techniques such as gel permeation, ion exchange, partition chromatography, affinity chromatography (including monoclonal antibody columns) or counter-current distribution.

The starting materials required for use in the chemical synthesis of peptides described above are known in the literature or can be prepared using known methods and known starting materials.

The peptides forming CDRs of the antigen binding site of antibodies or fragments thereof may also be prepared by recombinant DNA technology.

The various nucleotide sequences that encode the peptides forming the CDRs of the antigen binding site of antibodies or fragments can be determined using standard techniques.

The invention also provides a nucleic acid molecule including a sequence that is complementary to the sequence of the various nucleic acid molecules encoding the peptides according to the invention. A nucleic acid molecule that can hybridise to a molecule having one of the above described nucleotide sequences in high stringency conditions is particularly useful as the complementary strand of this nucleic acid molecule may well encode a peptide of the invention that is a variant. As is well known in the art, hybridisation of nucleic acid molecules may be controlled by the type of buffer used for hybridisation and the temperature of the buffer. “High stringency conditions” are conditions in which the buffer includes about 0.1×SSC, 0.1% SDS and the temperature is about 60° C.

The above described nucleic acid molecules can be obtained from genomic DNA, for example by PCR amplification, from a genomic library, from cDNA derived from mRNA, from a cDNA library, or by synthetically constructing the DNA sequence using synthetically derived nucleotides; (Sambrook et al., Molecular Cloning. A Laboratory Manual (2d ed.), Cold Spring Harbour laboratory, N.Y., 1989).

The nucleic acid molecule of the invention may be a deoxyribonucleotide, a ribonucleotide, a peptide nucleic acid or a combination thereof.

The invention also provides a vector or construct including a nucleic acid molecule of the invention.

The vector or construct is typically obtained by inserting a nucleic acid molecule of the invention into an appropriate plasmid or vector which can be used to transform a cell, for example, a host cell. In general, plasmid vectors containing replication and control sequences which are derived from species compatible with the host cell are used in connection with those hosts. The vector ordinarily carries a replication site, as well as sequences which encode proteins or peptides that are capable of providing phenotypic selection in transformed cells.

For example, E. coli may be transformed using pBR322, a plasmid derived from an E. coli species, see for example Mandel et al., J. Mol. Biol. 53: 154 (1970). Plasmid pBR322 contains genes for ampicillin and tetracycline resistance and thus provides for easy means for selection. Other vectors include different features such as different promoters, which are often important in expression. For example, plasmids pKK223-3, pDR720, and pPL-lambda represent expression vectors with the tac, trp, or P_{L} promoters that are currently available (Pharmacia Biotechnology).

A useful vector is pB0475. This vector contains origins of replication for phage and E. coli that allow it to be shuttled between such hosts, thereby facilitating both mutagenesis and expression, see for example, Cunningham et al., Science, 243: 1330-1336 (1989); U.S. Pat. No. 5,580,723. Other useful vectors are pR1T5 and pR1T2T (Pharmacia Biotechnology). These vectors contain appropriate promoters followed by the Z domain of protein A, allowing genes inserted into the vectors to be expressed as fusion proteins.

Other useful vectors can be constructed using standard techniques by combining the relevant traits of the vectors described above. Relevant traits include the promoter, the ribosome binding site, the decorsin or ornatin gene or gene fusion (the Z domain of protein A and decorsin or ornatin and its linker), the antibiotic resistance markers, and the appropriate origins of replication.

The invention also provides a cell including a vector or construct as described above. The host cell may be prokaryotic or eukaryotic.

Prokaryotes may be used for cloning and expressing a nucleic acid molecule of the invention to produce the peptide of the invention. For example, E. coli K12 strain 294 (ATCC No. 31446) may be used as well as E. coli B, E. coli X1776 (ATC No. 31537), and E. coli c600 and c600hfl, E. coli W3110 (F-, gama-, prototrophic/ATCC No. 27325), bacilli such as Bacillus subtilis, and other Enterobacteriaceae such as Salmonella typhimurium or Serratia marcesans, and various Pseudomonas species. When expressed by prokaryotes the peptide of the invention may contain an N-terminal methionine or a formyl methionine and may not be glycosylated. In the case of fusion proteins, the N-terminal methionine or formyl methionine may reside on the amino terminus of the fusion protein or the signal sequence of the fusion protein.

In addition to prokaryotes, eukaryotic organisms, such as yeast cultures, or cells derived from multicellular organisms may be used. In principle, any such cell culture is workable. However, interest has been greatest in vertebrate cells, and propagation-of vertebrate cells in culture (tissue culture) has become reproducible procedure, see for example, Tissue Culture, Academic Press, Kruse and Patterson, editors (173). Examples of such useful host cell lines are VERO and HeLa cells, Chinese Hamster Ovary (CHO) cells lines, W138, 293, BHK, COS-7 and MDCK cell lines.

The invention also provides a process for producing a peptide of the invention. The process includes maintaining a cell containing a nucleic acid molecule as described above, or a vector or construct as described above, in conditions for permitting the cell to produce the peptide.

The process may optionally include the step of recovering and or purifying the protein. Purification of the peptide is typically achieved using conventional procedures such as preparative HPLC (including reversed phase HPLC) or other known chromatographic techniques such as gel permeation, ion exchange, partition chromatography, affinity chromatography (including monoclonal antibody columns) or counter-current distribution.

The sequences of the various peptides forming the CDRs of the antigen binding sites of the antibodies and fragments thereof are useful as starting information to prepare affinity matured antigen binding sites. Generally speaking, affinity maturation requires rounds of selective mutation of hypervariable regions and screening on a determinant of interest.

A useful method for identification of a residue of a peptide forming a CDR as described above for amino acid substitution to generate a variant is called alanine scanning mutagenesis as described by Cunningham and Wells (1989) Science, 244:1081-1085. Here a residue or group of target residues are identified (eg charged residues such as Asn, Gln and Lys) and replaced by a neutral or negatively charged amino acid to affect the interaction of the amino acids with the surrounding environment. Those domains demonstrating functional sensitivity to the substitution then are refined by introducing further or other variations at or for the sites of substitution. Thus while the site for introducing an amino acid sequence variation is predetermined the nature of the mutation per se need not be predetermined. For example, to optimize the performance of a mutation at a given site, Ala scanning or random mutagenesis may be conducted at the target codon or region and the expressed peptide screened for the optimal combination of desired activity.

Phage display of protein or peptide libraries offers another methodology for the selection of peptide with improved or altered affinity, specificity, or stability (Smith, G, P, (1991) Curr Opin Biotechnol (2:668-673). High affinity proteins, displayed in a monovalent fashion as fusions with the M13 gene III coat protein (Clackson, T, (1994) et al, Trends Biotechnol 12:173-183), can be identified by cloning and sequencing the corresponding DNA packaged in the phagemid particles after a number of rounds of binding selection.

In further embodiments of the invention, the polypeptide or fragment thereof may comprise any combination of CDR1_{L}, CDR2_{L} and CDR3_{L} including CDR1_{L} and CDR2_{L}, CDR2_{L} and CDR3_{L}, CDR1_{L} and CDR3_{L}, and CDR1_{L}, CDR2_{L} and CDR3_{L}.

In other embodiments of the invention, the polypeptide or fragment thereof may comprise any combination of CDR1_{H}, CDR2_{H} and CDR3_{H} including CDR1_{H} and CDR2_{H}, CDR2_{H} and CDR3_{H}, CDR1_{H} and CDR3_{H}, and CDR1_{H}, CDR2_{H} and CDR3_{H}.

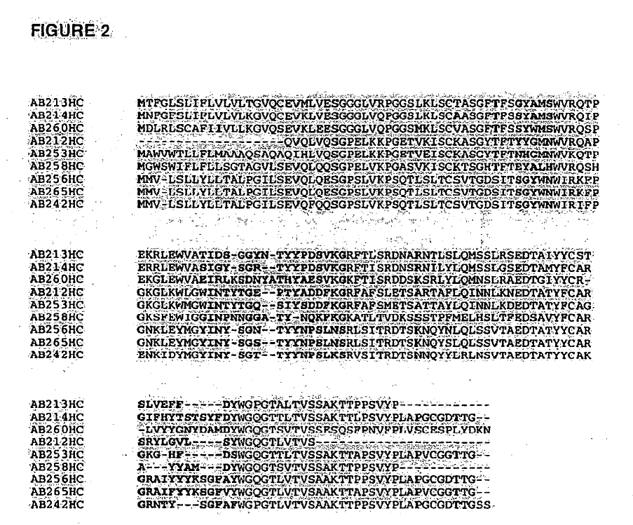

In another aspect there is provided an immune complex formed from the binding of an antibody or fragment thereof described above to a non functional P2X7 receptor, monomer or fragment thereof, or to a peptide shown in FIG. 2.

An immune complex is otherwise known as an antibody-antigen complex. This is formed when the antigen binding site of an antibody binds to an antigenic determinant or epitope. The determinant or epitope is generally of the same sequence or conformation as the immunogen against which the antibody was raised. The binding interaction is non covalent and typically comprised of ionic interactions, hydrogen bonding and hydrophobic interactions.

The immune complex is particularly important as detection of this in vitro or in vivo is indicative of presence of, or predisposition to a disease or condition including preneoplasia and neoplasia. These detection methods are described in more detail below.

The P2X7 receptor, monomer or fragment thereof included in the immune complex may have Pro210 in cis isomerisation.

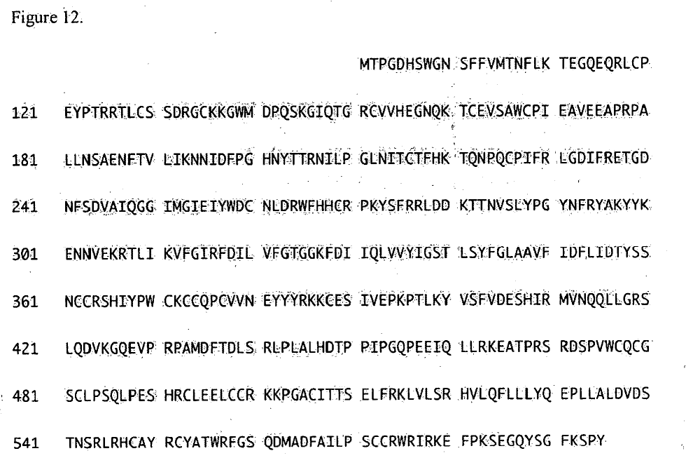

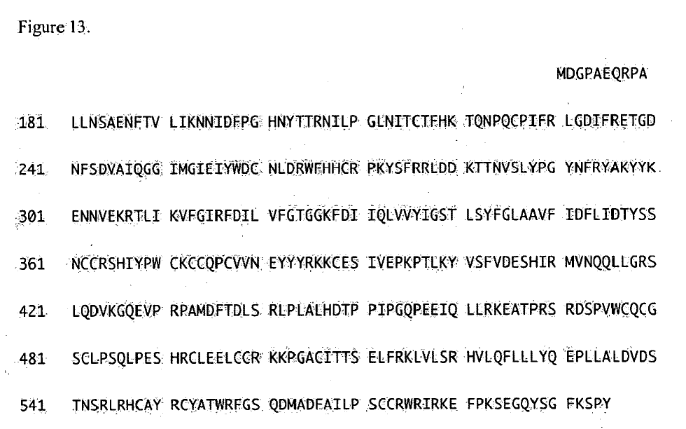

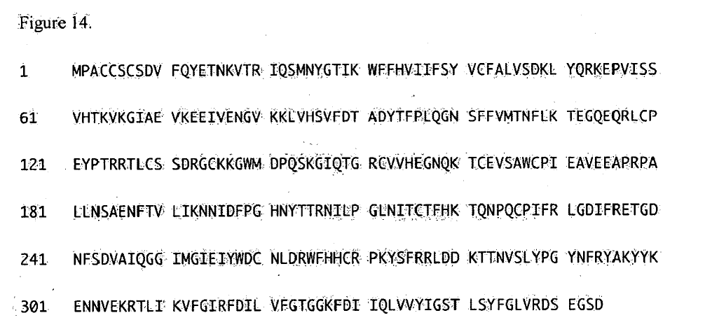

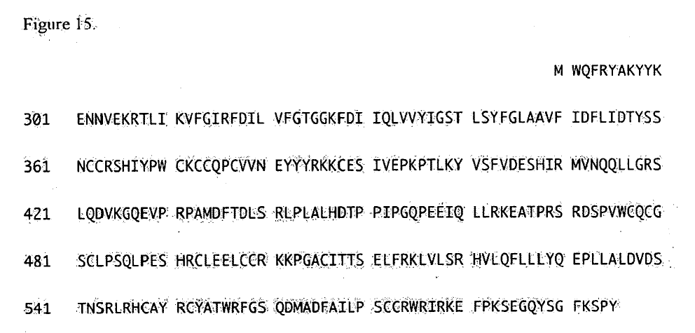

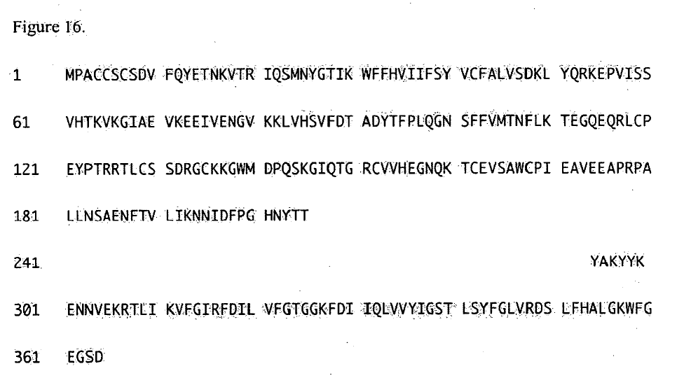

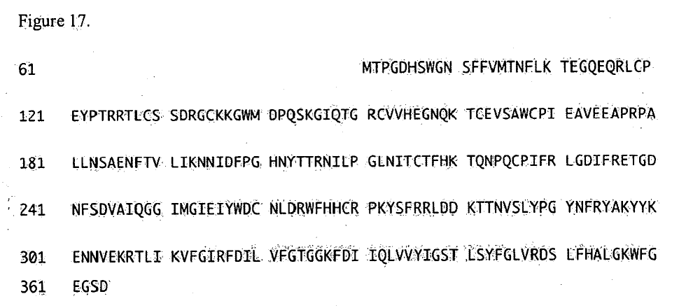

The P2X7 receptor, monomer or fragment thereof included in the immune complex may have an amino acid sequence as shown in any one of FIGS. 11 to 17 (SEQ ID NOS: 58 to 64) or fragment thereof.

The P2X7 receptor, monomer or fragment thereof included in the immune complex may have a molecular weight in the range of from about 15 to 80 kDa, not including the molecular weight of the antibody or antibody fragment. The total molecular weight depends on the whether the complex is formed from a whole antibody or fragment thereof.

The P2X7 receptor, monomer or fragment thereof included in the immune complex may lack a transmembrane domain.

The immune complex may be formed by binding a P2X7 receptor, monomer or fragment thereof located on a cell surface membrane, in a cytoplasm, in a nucleus or in extra-cellular fluid. The extra-cellular fluid may be blood, plasma, serum, lymph, urine, semen, saliva, sputum, ascites, faeces, uterine and vaginal secretions, bile, amniotic fluid, cerebrospinal fluid and organ and tissue flushings.

The antibody or antibody fragment included in the immune complex may be attached to a solid phase, such as a bead or a plate, so that the immune complex is attached to a solid phase when formed. Alternatively, the P2X7 receptor, monomer or fragment thereof included in the immune complex may be attached to a solid phase.

The antibody may be labelled for detection of formation of the immune complex.

The immune complex may further include an antibody or fragment thereof, such as a capture antibody for capture of the immune complex. The further antibody or fragment thereof may bind to the anti P2X7 receptor antibody. Also, the further antibody or fragment thereof may bind to the receptor or fragment thereof.

The further antibody or fragment thereof may be bound to a solid phase such as a phase described above.

The further antibody may be labelled for detection of formation of the immune complex. Examples of labels include fluorophores, dyes, isotopes etc.

In another aspect there is provided a method for determining whether a cell, tissue or extra cellular body fluid includes a non functional P2X7 receptor, monomer or fragment thereof including:
contacting a cell, tissue or extra cellular body fluid with an antibody or fragment thereof described above in conditions for forming an immune complex as described above, and
detecting whether an immune complex has been formed,
   wherein detection of an immune complex determines that a cell, tissue or extra-cellular body fluid includes a non functional P2X7 receptor, monomer or fragment thereof.

In other embodiments there is provided a use of an antibody or fragment thereof described above in the manufacture of means for determining whether a cell, tissue or extra-cellular body fluid contains a P2X7 receptor, monomer or fragment thereof.

The presence of a given protein, or level of expression of a given protein in a host cell, tissue or extra-cellular body fluid can be detected by any number of assays. Examples include immunoassays, chromatography and mass spectrometry.

Immunoassays, i.e. assays involving an element of the immune system are particularly preferred. These assays may generally be classified into one of:

(i) assays in which purified antigen is used to detect an antibody in host serum. For example, purified antigen is bound to solid phase by adsorption or indirectly through another molecule and host serum is applied followed by another antibody for detecting presence or absence of host antibody;

(ii) assays in which purified antigen is used to detect immune cells, such as T and B lymphocytes. For example, peripheral white cells are purified from a host and cultured with purified antigen. The presence or absence of one or factors indicating immunity are then detected. Other examples include assays that measure cell proliferation (lymphocyte proliferation or transformation assays) following exposure to purified antigen, and assays that measure cell death (including apoptosis) following exposure to purified antigen;

(iii) assays in which purified antibody specific for antigen is used to detect antigen in the host. For example, purified antibody is bound to solid phase, host tissue is then applied followed by another antibody specific for the antigen to be detected. There are many examples of this approach including ELISA, RIA;

(iv) assays in which a purified anti-idiotypic antibody is used to detect host antibody. For example, anti-idiotypic antibody is adsorbed to solid phase, host serum is added and anti-Fc antibody is added to bind to any host antibodies having been bound by the anti-idiotypic antibody.

The immunoassays can be applied in vitro or in vivo.

In one embodiment, the disease is typically a cancer such as carcinoma, sarcoma, lymphoma, or leukemic nodule. Carcinomas that may be detected include, but not limited to, prostate, breast, skin, lung, cervix, uterus, stomach, oesophagus, bladder, liver, renal and colon cancers.

Whilst any body fluid can be used to detect any of these diseases, some body fluids may be more appropriate than others to detect certain diseases, for example urine may be more appropriate to detect prostate, ovarian and bladder cancer and blood for detecting blood cancers such as lymphoma.

In certain embodiments, cancer is selected from the group consisting of prostate cancer, invasive breast cancer, melanoma, adenocarcinoma of the bowel, serous ovarian cancer, squamous cell cancer of the cervix, endometrial cancer, small cell lung cancer, hepatocellular carcinoma, transitional cell carcinoma of the bladder, gastrointestinal stromal tumour, endometrial stromal tumour, pituitary cancer, mesothelioma, Hodgkin's lymphoma and thyroid papillary carcinoma.

In another aspect there is provided a kit or composition for determining whether a cell, tissue or extra-cellular body fluid contains a non functional P2X7 receptor, monomer or fragment thereof including:
an antibody or fragment thereof described above for forming an immune complex with a non functional P2X7 receptor, monomer or fragment thereof and optionally;
a further antibody for binding to the antibody or fragment thereof or the non functional P2X7 receptor, monomer or fragment thereof;
written instructions for use of the kit in a method described above.

Kits are provided which contain the necessary reagents to carry out the assays of the present invention. The kit may include one or more compartments, each to receive one or more containers such as: (a) a first container comprising one of the components of the present invention described above; and (b) one or more other containers comprising one or more of the following: wash reagents, reagents capable of detecting presence of the antibody or peptide.

The containers allow one to efficiently transfer reagents from one compartment to another compartment such that the samples and reagents are not cross-contaminated, and the agents or solutions of each container can be added in a quantitative fashion from one compartment to another.

The kit typically contains containers which may be formed from a variety of materials such as glass or plastic, and can include for example, bottles, vials, syringes, and test tubes. A label typically accompanies the kit, and includes any writing or recorded material, which may be electronic or computer readable form (e.g., disk, optical disc, or tape) providing instructions or other information for used of the contents of the kit. The label indicates that the formulation is used for diagnosing or treating the disorder of choice.

One skilled in the art will readily recognize that the disclosed antibodies and peptides of the present invention can be readily incorporated into one of the established kit formats which are well known in the art.

In other embodiments. there is provided a pharmaceutical composition including an antibody or fragment thereof as described above together with a pharmaceutically acceptable carrier, diluent or excipient.

In the preparation of the pharmaceutical compositions comprising the antibodies or peptides described in the teachings herein, a variety of vehicles and excipients and routes of administration may be used, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A.H. ed., Washington D.C., American Pharmaceutical Association (2000); hereby incorporated by reference in their entirety.

The pharmaceutical compositions will generally comprise a pharmaceutically acceptable carrier and a pharmacologically effective amount of the antibodies or peptides, or mixture of antibodies or mixture of peptides, or suitable salts thereof.

The pharmaceutical composition may be formulated as powders, granules, solutions, suspensions, aerosols, solids, pills, tablets, capsules, gels, topical creams, suppositories, transdermal patches, and other formulations known in the art.

For the purposes described herein, pharmaceutically acceptable salts of the antibodies and peptides is intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts include organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any standard pharmaceutically accepted carriers known to those of ordinary skill in the art in formulating pharmaceutical compositions. Thus, the antibodies or peptides, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or as solid formulations in appropriate excipients.

The pharmaceutical compositions will often further comprise one or more buffers (e.g., neutral buffered saline or phosphate buffered saline), carbohydrates (e.g., glucose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, adjuvants (e.g., aluminium hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

While any suitable carrier known to those of ordinary skill in the art may be employed in the compositions of this invention, the type of carrier will typically vary depending on the mode of administration. Antibody and peptide compositions may be formulated for any appropriate manner of administration, including for example, oral, nasal, mucosal, intravenous, intraperitoneal, intradermal, subcutaneous, and intramuscular administration.

For parenteral administration, the compositions can be administered as injectable dosages of a solution or suspension of the substance in a physiologically acceptable diluent with a pharmaceutical carrier that can be a sterile liquid such as sterile pyrogen free water, oils, saline, glycerol, polyethylene glycol or ethanol. Additionally, auxiliary substances, such as wetting or emulsifying agents, surfactants, pH buffering substances and the like can be present in compositions.

Other components of pharmaceutical compositions are those of petroleum, animal, vegetable, or synthetic origin, for example, non-aqueous solutions of peanut oil, soybean oil, corn oil, cottonseed oil, ethyl oleate, and isopropyl myristate. Antibodies and peptides can be administered in the form of a depot injection or implant preparation which can be formulated in such a manner as to permit a sustained release of the active ingredient. An exemplary composition comprises antibody at 5 mg/ml, formulated in aqueous buffer consisting of 50 mM L-histidine, 150 mM NaCl, adjusted to pH 6.0 with HCI.

Typically, the compositions are prepared as injectables, either as liquid solutions or suspensions; solid or powder forms suitable for reconstitution with suitable vehicles, including by way example and not limitation, sterile pyrogen free water, saline, buffered solutions, dextrose solution, etc., prior to injection. The preparation also can be emulsified or encapsulated in liposomes or micro particles such as polylactide, polyglycolide, or copolymers.

The pharmaceutical compositions described herein may be presented in unit-dose or multi-dose containers, such as sealed ampoules or vials. Such containers are typically sealed in such a way to preserve the sterility and stability of the formulation until use. In general, formulations may be stored as suspensions, solutions or emulsions in oily or aqueous vehicles, as indicated above.

Alternatively, a pharmaceutical composition may be stored in a freeze-dried condition requiring only the addition of a sterile liquid carrier immediately prior to use.

In related embodiments there is provided a method of treatment of a disease characterised by the expression of a non ATP-binding P2X7 receptor, monomer or fragment thereof including the step of providing an antibody or fragment thereof as described above, or a peptide as described above to an individual requiring said treatment.

Methods of immunotargeting cancer cells using antibodies or antibody fragments are well known in the art. U.S. Pat. No. 6,306,393 describes the use of anti-CD22 antibodies in the immunotherapy of B-cell malignancies, and U.S. Pat. No. 6,329,503 describes immunotargeting of cells that express serpentine transmembrane antigens. Antibodies described herein (including humanized or human monoclonal antibodies or fragments or other modifications thereof, optionally conjugated to cytotoxic agents) can be introduced into a patient such that the antibody binds to cancer cells and mediates the destruction of the cells and the tumor and/or inhibits the growth of the cells or the tumor.

Without intending to limit the disclosure, mechanisms by which such antibodies can exert a therapeutic effect may include complement-mediated cytolysis, antibody-dependent cellular cytotoxicity (ADCC) modulating the physiologic function of the tumor antigen, inhibiting binding or signal transduction pathways, modulating tumor cell differentiation, altering tumor angiogenesis factor profiles, modulating the secretion of immune stimulating or tumor suppressing cytokines and growth factors, modulating cellular adhesion, and/or by inducing apoptosis. And internalisation of the antibody.

The antibodies can also be conjugated to toxic or therapeutic agents, such as radioligands or cytosolic toxins, and may also be used therapeutically to deliver the toxic or therapeutic agent directly to tumor cells.

By “treatment” herein is meant therapeutic or prophylactic treatment, or a suppressive measure for the disease, disorder or undesirable condition. Treatment encompasses administration of the subject antibodies in an appropriate form prior to the onset of disease symptoms and/or after clinical manifestations, or other manifestations, of the disease to reduce disease severity, halt disease progression, or eliminate the disease. Prevention of the disease includes prolonging or delaying the onset of symptoms of the disorder or disease, preferably in a subject with increased susceptibility to the disease.

The therapeutic preparations can use nonmodified antibodies or antibodies conjugated with a therapeutic compound, such as a toxin or cytotoxic molecule, depending on he functionality of the antibody. Generally, when nonmodified antibodies are used, they will typically have a functional Fc region. By “functional Fc region” herein is meant a minimal sequence for effecting the biological function of Fc, such as binding to Fc receptors, particularly FcyR (e.g., Fc&#947;RI, FcyRII, and Fc&#947; RIII).

Without being bound by theory, it is believed that the Fc region may affect the effectiveness of anti-tumor monoclonal antibodies by binding to Fc receptors immune effector cells and modulating cell mediated cytotoxicity, endocytosis, phagocytosis, release of inflammatory cytokines, complement mediate cytotoxicity, and antigen presentation. In this regard, polyclonal antibodies, or mixtures of monoclonals will be advantageous because they will bind to different epitopes and thus have a higher density of Fc on the cell surface as compared to when a single monoclonal antibody is used. Of course, to enhance their effectiveness in depleting targeted cells, or where nonmodified antibodies are not therapeutically effective, antibodies conjugated to toxins or cytotoxic agents may be used.

The antibody compositions may be used either alone or in combination with other therapeutic agents to increase efficacy of traditional treatments or to target abnormal cells not targeted by the antibodies. Combining the antibody therapy method with a chemotherapeutic, radiation or surgical regimen may be preferred in patients that have not received chemotherapeutic treatment, whereas treatment with the antibody therapy may be indicated for patients who have received one or more chemotherapies. Additionally, antibody therapy can also enable the use of reduced dosages of concomitant chemotherapy, particularly in patients that do not tolerate the toxicity of the chemotherapeutic agent very well. Furthermore, treatment of cancer patients with the antibody with tumors resistant to chemotherapeutic agents might induce sensitivity and responsiveness to these agents in combination.

In one aspect, the antibodies are used adjunctively with therapeutic cytotoxic agents, including, by way of example and not limitation, busulfan, thioguanine, idarubicin, cytosine arabinoside, 6-mercaptopurine, doxorubicin, daunorubicin, etoposide, and hydroxyurea. Other agents useful as adjuncts to antibody therapy are compounds directed specifically to the abnormal cellular molecule found in the disease state. These agents will be disease specific. For example, for treating chronic myeloid leukemia arising from BCR-ABL activity, one class of useful compounds are inhibitors of abl kinase activity, such as Imatinib, an inhibitor of bcr-abl kinase, and antisense oligonucleotides against bcr (e.g., Oblimersen). Other agents include, among others, interferon-alpha, humanized anti-CD52, deacetylase inhibitor FR901228 (depsipeptide), and the like.

The amount of the compositions needed for achieving a therapeutic effect will be determined empirically in accordance with conventional procedures for the particular purpose. Generally, for administering the compositions ex vivo or in vivo for therapeutic purposes, the compositions are given at a pharmacologically effective dose. By “pharmacologically effective amount” or “pharmacologically effective dose” is an amount sufficient to produce the desired physiological effect or amount capable of achieving the desired result, particularly for treating or retreating the disorder or disease condition, including reducing or eliminating one or more symptoms or manifestations of the disorder or disease.

As an illustration, administration of antibodies to a patient suffering from prostate cancer provides a therapeutic benefit not only when the underlying disease is eradicated or ameliorated, but also when the patient reports a decrease in the severity or duration of the symptoms associated with the disease. Therapeutic benefit also includes halting or slowing the progression of the underlying disease or disorder, regardless of whether improvement is realized.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery of the agent, half-life of the agents, and efficacy of the agents. Stage of the disease to consider includes whether the disease is acute or chronic, relapsing or remitting phase, and the progressiveness of the disease. Determining the dosages and times of administration for a therapeutically effective amount are well within the skill of the ordinary person in the art.

For any compositions of the present disclosure, the therapeutically effective dose is readily determined by methods well known in the art. For example, an initial effective dose can be estimated from cell culture or other in vitro assays. For example, Sliwkowsky, M X et al., Semin. Oncol. 26. (suppl. 12) 60-70 (1999) describes in vitro measurements of antibody dependent cellular cytoxicity. A dose can then be formulated in animal models to generate a circulating concentration or tissue concentration, including that of the IC50 as determined by the cell culture assays.

In addition, the toxicity and therapeutic efficacy are generally determined by cell culture assays and/or experimental animals, typically by determining a LD50 (lethal dose to 50% of the test population) and ED50 (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein. Guidance is also found in standard reference works, for example Fingl and Woodbury, General Principles In: The Pharmaceutical Basis of Therapeutics pp. 1-46 (1975), and the references cited therein.

To achieve an initial tolerizing dose, consideration is given to the possibility that the antibodies may be immunogenic in humans and in non-human primates. The immune response may be biologically significant and may impair the therapeutic efficacy of the antibody even if the antibody is partly or chiefly comprised of human immunoglobulin sequences such as, for example, in the case of a chimeric or humanized antibody. Within certain embodiments, an initial high dose of antibody is administered such that a degree of immunological tolerance to the therapeutic antibody is established.

The tolerizing dose is sufficient to prevent or reduce the induction of an antibody response to repeat administration of the committed progenitor cell specific antibody.

Preferred ranges for the tolerizing dose are between 10 mg/kg body weight to 50 mg/kg body weight, inclusive. More preferred ranges for the tolerizing dose are between 20 and 40 mg/kg, inclusive. Still more preferred ranges for the tolerizing dose are between 20 and 25 mg/kg, inclusive.

Within these therapeutic regimens, the therapeutically effective dose of antibodies is preferably administered in the range of 0.1 to 10 mg/kg body weight, inclusive. More preferred second therapeutically effective doses are in the range of 0.2 to 5 mg/kg body weight, inclusive. Still more preferred therapeutically effective doses are in the range of 0.5 to 2 mg/kg, inclusive. Within alternative embodiments, the subsequent therapeutic dose or doses may be in the same or different formulation as the tolerizing dose and/or may be administered by the same or different route as the tolerizing dose.

For the purposes of this invention, the methods of administration are chosen depending on the condition being treated, the form of the subject antibodies, and the pharmaceutical composition.

Administration of the antibody compositions can be done in a variety of ways, including, but not limited to, continuously, subcutaneously, intravenously, orally, topically, transdermal, intraperitoneal, intramuscularly, and intravesically. For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, intraperitoneal administration can be accompanied by intravenous injections. Preferably the therapeutic doses are administered intravenously, intraperitonealy, intramuscularly, or subcutaneously.

The compositions may be administered once or several times. In some embodiments, the compositions may be administered once per day, a few or several times per day, or even multiple times per day, depending upon, among other things, the indication being treated and the judgement of the prescribing physician.

Administration of the compositions may also be achieved through sustained release or long-term delivery methods, which are well known to those skilled in the art. By “sustained release” or “long term release” as used herein is meant that the delivery system administers a pharmaceutically therapeutic amount of subject compounds for more than a day, preferably more than a week, and most preferable at least about 30 days to 60 days, or longer. Long term release systems may comprise implantable solids or gels containing the antibodies, such as biodegradable polymers described above; pumps, including peristaltic pumps and fluorocarbon propellant pumps; osmotic and mini-osmotic pumps; and the like.

The method of the invention contemplates the administration of single monoclonal antibodies and any antibody that recognizes the particular antigens recognized by these antibodies, as well as combinations, of different mAbs. Two or more monoclonal antibodies may provide an improved effect compared to a single antibody. Alternatively, a combination of an antibody with an antibody that binds a different antigen may provide an improved effect compared to a single antibody. Such mAb cocktails may have certain advantages inasmuch as they contain mAbs, which exploit different effector mechanisms or combine directly cytotoxic mAbs with mAbs that rely on immune effector functionality. Such mAbs in combination may exhibit synergistic therapeutic effects.

In order that the nature of the present invention may be more clearly understood, preferred forms thereof will now be described with reference to the following non-limiting examples.

### EXAMPLE 1

Identification of CDR Sequences from Hybridomas

The preferred animal system for generating hybridomas is the murine system. Immunization protocols and techniques for isolation of immunized splenocytes for fusion are well known in the art. Fusion cell partners (e.g., murine myeloma cell lines SP2/0, NSO, NS1, rat myeloma Y3, rabbit myeloma 240E 1, human K6H6), fusion and screening procedures are also well known in the art.

### (i) Hybridoma Generation

B cell-myeloma cell hybridomas were generated using splenocytes from immunised mice as follows:

Immunization: BALB/c mice (female, 8-10 weeks of age at first injection, CSIRO animal facility, North Ryde, Australia) were immunized with conjugate comprising human P2X₇ 200-216 linked to diphtheria toxoid (at a conjugation ratio of approximately 11:1) emulsified in adjuvant. The initial immunization was performed with conjugate in Montanide-QuilA-DEAE dextran, 4×50 μL injections per mouse (2 intramuscularly and 2 subcutaneously), 250 μg/mL. Subsequent immunizations were performed at 2-4 weekly intervals with conjugate in ImmunEasy™ adjuvant (Qiagen), 2×50 μL injections per mouse (1 intramuscularly and 1 subcutaneously), 200 μg/mL. After at least 3 immunization cycles, mice were injected with 20 μg conjugate intravenously in sterile phosphate-buffered saline.

Hybridoma generation: Four to five days after the intravenous boost, spleens were recovered and spleen cell suspensions prepared. Spleen cells were fused with SP2/0-Ag14 myeloma cells by mixing at a ratio of 5:1 in a 50% solution of polyethylene glycol 1500 (Roche Cat No. 783 641) in serum-free medium (RPMI with 2 mM L-glutamine, 1 mM sodium pyruvate, 50IU/mL penicillin and 50 μg/mL streptomycin; Gibco). After incubation at 37° C. for 2 minutes, the cell suspension was diluted in serum-free medium, and pelleted by centrifugation (8 minutes, 70×g). Cells were cultivated in RPMI medium (as above, supplemented with 10% foetal bovine serum, HAT (Gibco) and 100U/mL recombinant murine IL-6 (Peprotech). After 12-14 days, cell culture supernatants were assayed for reactivity by ELISA with solid-phase human P2X₇ 200-216 linked to bovine serum albumin (at a conjugation ratio of approximately 11:1). Positive wells were subcloned at limiting dilution.

(ii) Determination of Antibody Sequences from Hybridomas

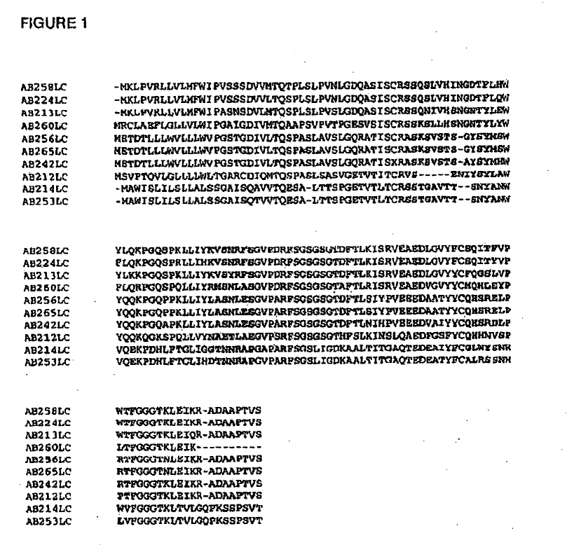

Antibody sequences for both the light and heavy chain variable regions were determined from antibodies produced by hybridomas of the invention according to the following methods. A total of 10 light chain (FIG. 1) and 9 heavy chain (FIG. 2) variable sequences were obtained.

### Method 1

Total RNA (tRNA) was extracted from 5×10⁶ viable hybridoma cells using TRIzol reagent (Invitrogen) according to the manufacturer's instructions. Following quantitation, poly-A⁺ RNA was isolated using Oligotex mRNA mini columns (QIAgen) according to the manufacturer's instructions. Once quantified again, 500 ng of poly-A⁺ RNA was used as a template for first strand cDNA synthesis using a range of isotype specific primer and Omniscript reverse transcriptase kit (QIAgen). Following synthesis the first strand cDNA was purified using a QIAquick PCR purification column (QIAgen) and Poly-G tailed using terminal transferase and dGTP (New England Biolabs).

The tailed first strand cDNA was again purified using a QIAquick PCR purification column and used as a template for high fidelity PCR using Vent polymerase (New England Biolabs).

Typically PCR reactions were over 40 cycles using an isotype specific reverse primer and an oligo (dC) forward primer.

The resulting PCR fragments were resolved through a 1.5% agarose gel and major bands of ˜550 bp for V_{L} and ˜600 bp for V_{H} were extracted from the gel and purified using MinElute PCR purification kit (QIAgen) and ligated into the pPCR-Script Amp SK(+) vector using the PCR-Script cloning kit (Stratagene) all according to manufacturer's instructions. Ligation mixes were transformed into XL10-Gold ultracompetent E. coli cells. and plasmids were extracted from overnight culture of single colonies using QIAquick Miniprep columns (QIAgen) and quantified. 100 to 500 ng were mixed in duplicate with 6.4 pmol of either USP (5′-GTAAAACGACGGCCAGT-3′) or RSP (5′-GGAAACAGCTATGACCATG-3′) primer and submitted to cycle sequencing. Sequencing data was analysed and consensus sequences determined using Clone Manager Professional Suite software (Sci Ed Software).

### Method 2

tRNA was extracted from 1×10⁷ to 1×10⁸ viable hybridoma cells using RNeasy Mini or Midi columns (QIAgen) according to the manufacturer's instructions. Following quantitation the tRNA was used as a template for first strand cDNA synthesis using an oligo(dT) primer and Superscript II Reverse Transcriptase (Invitrogen) according to manufacturer's instructions. Finally the tRNA was degraded using RNase H and the remaining single stranded cDNA tagged with a poly-G tail using terminal transferase and dGTP (Roche).

PCR reactions where performed using Herculase (Stratagene), a high fidelity polymerase blend. In each case an oligo (dC) was used as the forward primer with an isotype specific reverse primer. Following 30 cycles PCR reactions were cleaned up using QIAquick PCR clean up columns (QIAgen) and quantified on a Biophotometer (Eppendorf).

100 to 500 ng of PCR product were mixed with 6.4 pmol of the relevant isotype specific primer and submitted to cycle sequencing using BigDye v3.1 chemistry (AppliedBiosystems). Sequencing reactions were set up in duplicate and electrophoretograms resolved on either ABI PRISM 377 or 3700 DNA Analysers. Following alignment of derived sequence and manual correction of aberrant base calling a sequence specific forward sequencing primer was designed and used to resubmit the sample for DNA sequencing in the forward orientation. Once four sequences (2 forward and 2 reverse) were obtained the sequence of the antibodies variable region was confirmed.

Where insufficient quantity of PCR fragment was obtained from the PCR reaction for sequencing directly, the product was incubated in the presence of Taq polymerase and dNTPs to add overhanging A bases. This was then cloned into pGemT-Easy (Promega) and transformed into competent Top 10 E. coli cells (Invitrogen). Plasmids were extracted from overnight culture of single colonies using QIAquick Miniprep columns (QIAgen) and quantified. 100 to 500 ng were mixed in duplicate with 6.4 pmol of either pUC3 forward or pUC3 reverse primer and submitted to cycle sequencing. Sequencing data was analysed and consensus sequences determined using ContigExpress software, part of Vector NTi (Invitrogen).

The variable heavy and light chain region sequences for antibodies 213, 214, 253, 256, 258 and 265 were obtained using Method 1, whereas the sequences for antibodies 212, 224, 242 and 260 were obtained using Method 2.

(iii) Sequence Alignment

A total of 10 light chain and 9 heavy chain variable region sequences obtained from antibodies 212, 213, 214, 224, 242, 253, 256, 258, 260 and 265 were aligned using ClustalW based on sequence similarity (www.ebi.ac.uk/clustalw/index.html). The aligned sequences are shown in FIGS. 1 and 2; the light chain variable region sequences are shown in FIG. 1 and the heavy chain variable region sequences are shown in FIG. 2. The three CDRs for each variable chain region (CDR1_{L}, CDR2_{L}, CDR3_{L}, CDR1_{H}, CDR2_{H} and CDR3_{H}) were then identified according to Kabat's rules (www.bioinf.org.uk/abs/#cdrdef), and are indicated in bold in FIGS. 1 and 2.

### EXAMPLE 2

### Determination of Germline Antibody CDR Sequences

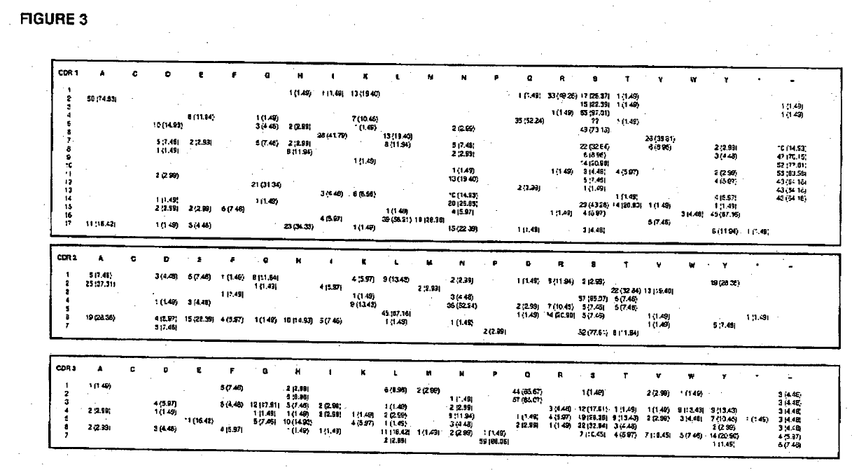

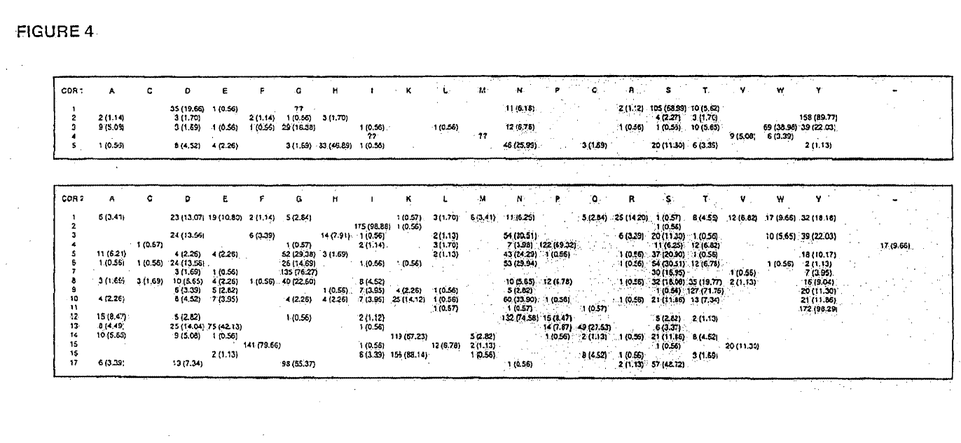

Murine germline heavy and light chain CDR sequences were obtained from The Antibody Group (http://www.ibt.unam.mx/vir/V_mice.html). A total of 177 heavy chain and 67 light chain CDR sequences were mapped by calculating the number and frequency of occurrence of each amino acid at each position. The results are presented in FIG. 3 for the light chain CDR1_{L}, CDR2_{L} and CDR3_{L} germline sequences, and FIG. 4 for the heavy chain CDR1_{H} and CDR2_{H} germline sequences.

In FIG. 3, the data presented for position 1 of CDR1_{L} shows that histidine occurs only once in the 67 light chain germline sequences analysed, representing a frequency of 1.49% (i.e. 1 (1.49%) in FIG. 3). The other amino acids found at position 1 of the light chain CDR1_{L} germline sequences are isoleucine (1 (1.49%)); lysine (13 (19.40%)), glutamine (1 (1.49%)); arginine (33 (49.25%)), serine (17 (25.37%)); and threonine (I (1.49%)). In FIGS. 3 and 4, the “−” indicates where an amino acid is missing in the germline sequence leading to a shorter CDR. Similarly “*” indicates where the germline sequence contains a stop codon at the genomic DNA sequence level.

All 177 heavy chain germline sequences terminate before the CDR3_{H} region of the antibody. This is most likely due to the fact that CDR3_{H} is created from V, D and J regions of genomic DNA, and is therefore extremely variable. As a result no attempt was made to assess the distribution and frequency of amino acids within the limited heavy chain CDR3_{H} of the antibody sequences.

### EXAMPLE 3

### Analysis of Germline and Hybridoma CDR Sequences

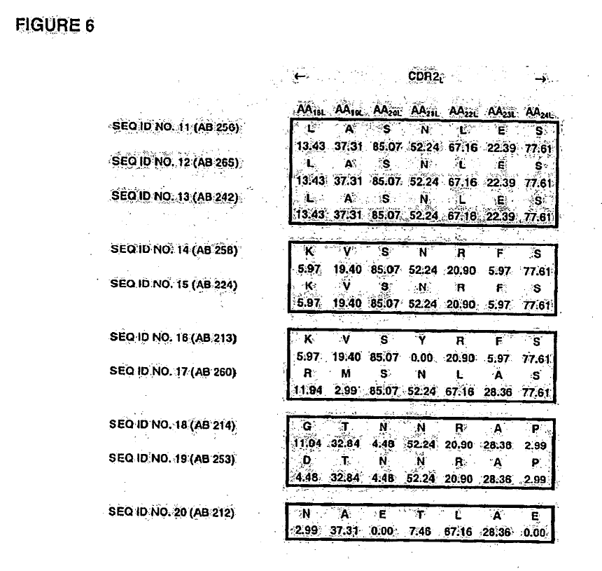

Using the information in Table 1, each of the CDR sequences identified in Example 1 was aligned based on similarity of their amino acid side chains. These sequences are represented in FIGS. 5-10 for the light chain CDR1_{L} (FIG. 5), light chain CDR2_{L} (FIG. 6), light chain CDR3_{L} (FIG. 7), and heavy chain CDR1_{H} (FIG. 8), heavy chain CDR2_{H} (FIG. 9) and heavy chain CDR3_{H} (FIG. 10) sequences. Note that each of the CDR sequences have also been assigned a sequence identifier (e.g. the light chain CDR1_{L} sequence derived from antibody 256 has been assigned SEQ ID NO. 1).

Also presented for FIGS. 5-9 are the values indicating the frequency of occurrence of the identical amino acid at the same position for the germline sequences (i.e. those values calculated according to Example 2; also see FIGS. 3 and 4). For example, referring to FIG. 5, threonine (T) which occurs at position 10 of CDR1_{L}, SEQ ID NO. 1, occurs only 5.97% of the time in germline sequences at the same position. Similarly, serine (S) at position 11 occurs only 7.46% of the time in germline sequences at the same position. Thus, the probability of finding a threonine followed by a serine at positions 10 and 11 of light chain CDR1_{L} sequences is extremely low.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

It will be understood that the invention disclosed and defined in this specification extends to all alternative combinations of two or more of the individual features mentioned or evident from the text or drawings. All of these different combinations constitute various alternative aspects of the invention.

1. A recombinant or synthetic antibody or fragment thereof, said antibody or fragment thereof including three complementarity determining regions (CDR1_{L}, CDR2_{L} and CDR3_{L}) for forming an antigen binding site that is capable of binding to a non functional P2X7 receptor but not capable of binding to a functional P2X7 receptor.

2. The antibody or fragment according to claim 1 wherein the CDR1_{L} includes a peptide having a sequence defined by the following formula:
AA_{1L} AA_{2L} AA_{3L} AA_{4L} AA_{5L} AA_{6L} AA_{7L} AA_{8L} AA_{9L} AA_{10L} AA_{11L} AA_{12L} AA_{13L} AA_{14L} AA_{15L} AA_{16L} AA_{17L}
wherein:
AA_{1L} is R; AA_{2L} is A, S or V; AA_{3L} is S; AA_{4L} is K, Q, T or E; AA_{5L} is S, N or G; AA_{6L} is V, L, I or A; AA_{7L} is S, V, L or absent; AA_{8L} is H or absent; AA_{9L} is I, S, T or absent; AA_{10L} is T or absent; AA_{11L} is S, N or absent, AA_{12L} is G, A or absent; AA_{13L} is Y, D, N or absent; AA_{14L} is S, T or N; AA_{15L} is Y or F; AA_{16L} is M, L or A; and AA_{17L} is S, H, Q, E, Y, N or A.

3. The antibody or fragment according to claim 2 wherein the peptide forming the CDR1_{L} is selected from the group consisting of: SEQ ID NO.1, SEQ ID NO.2, SEQ ID NO.3, SEQ ID NO.4, SEQ ID NO.5, SEQ ID NO.6, SEQ ID NO.7, SEQ ID NO.8, SEQ ID NO.9, and SEQ ID NO.10

4. The antibody or fragment according to claims wherein the CDR2_{L} includes a peptide having a sequence defined by the following formula:
AA_{18L} AA_{19L} AA_{20L} AA_{21L} AA_{22L} AA_{23L} AA_{24L}
wherein:
AA_{18L} is L, K, R, G, D or N; AA_{19L} is A, V, M or T; AA_{20L} is S, N or E; AA_{21L} is N, Y or T; AA_{22L} is L or R; AA_{23L} is E, F or A; and AA_{24L} is S, P or E.

5. The antibody or fragment according to claim 4 wherein the peptide forming the CDR2_{L} is selected from the group consisting of: SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20.

6. The antibody or fragment according to claim 1 wherein the CDR3_{L} includes a peptide having a sequence defined by the following formula:
AA_{25L}AA_{26L}AA_{27L}AA_{28L}AA_{29L}AA_{30L}AA_{31L}AA_{32L}AA_{33L}
wherein:
AA_{25L} is Q, S, F, M, A or G; AA_{26L} is H, Q or L; AA_{27L} is S, I, G, H, W or R; AA_{28L} is R, T, S, L, Y or N; AA_{29L} is E, D, F, L, S or V; AA_{30L} is L, V, Y, N or S; AA_{31L} is P or H; AA_{32L} is R, W, L or P; and AA_{33L} is T or V.

7. The antibody or fragment according to claim 6 wherein the peptide forming the CDR3_{L} is selected from the group consisting of: SEQ ID NO. 21, SEQ ID NO. 22, SEQ ID NO. 23, SEQ ID NO. 24, SEQ ID NO. 25, SEQ ID NO. 26, SEQ ID NO. 27, SEQ ID NO. 28, SEQ ID NO. 29 and SEQ ID NO. 30.

8. A recombinant or synthetic antibody or fragment thereof, said antibody or fragment thereof including three complementarity determining regions (CDR1_{H}, CDR2_{H} and CDR3_{H}) for forming an antigen binding site that is capable of binding to a non functional P2X7 receptor but not capable of binding to a functional P2X7 receptor.

9. The antibody or fragment according to claim 8 wherein the CDR1_{H} includes a peptide having a sequence defined by the following formula:
AA_{1H} AA_{2H} AA_{3H} AA_{4H} AA_{5H}
wherein:
AA_{1H} is S, E, G, Y or N; AA_{2H} is G, Y or H; AA_{3H} is Y, A, W or G; AA_{4H} is W, L or M; and AA_{5H} is N, H or S.

10. The antibody or fragment according to claim 9 wherein the peptide forming the CDR1_{H} is selected from the group consisting of: SEQ ID NO. 31, SEQ ID NO. 32, SEQ ID NO. 33, SEQ ID NO. 34, SEQ ID NO. 35, SEQ ID NO. 36, SEQ ID NO. 37, SEQ ID NO. 38 AND SEQ ID NO. 39.

11. The antibody or fragment according to claim 8 to 10 wherein the CDR2_{H} includes a peptide having a sequence defined by the following formula:
AA_{6H} AA_{7H} AA_{8H} AA_{9H} AA_{10H} AA_{11H} AA_{12H} AA_{13H} AA_{14H} AA_{15H} AA_{16H} AA_{17H} AA_{18H} AA_{19H} AA_{20H} AA_{21H} AA_{22H} AA_{23H} AA_{24H}
wherein:
AA_{6H} is Y, G, T, S, E or W; AA_{7H} is I; AA_{8H} is N, D, G or R; AA_{9H} is absent, P, S, L or T; AA_{10H} is K or absent; AA_{11H} is S or absent; AA_{12H} is Y, N, G or D; AA_{13H} is S, N, G or T; AA_{14H} is G or Y; AA_{15H} is N, S, T, G, R, A, E or Q; AA_{16H} is T, A, P or S; AA_{17H} is Y, T, H or I; AA_{18H} is Y; AA_{19H} is N, P, A or S; AA_{20H} is P, Q, D or E; AA_{21H} is S, K or D; AA_{22H} is L, F or V; AA_{23H} is N or K; and AA_{24H} is S or G.

12. The antibody or fragment according to claim 11 wherein the peptide forming the CDR2_{H} is selected from the group consisting of: SEQ ID NO. 40, SEQ ID NO. 41, SEQ ID NO. 42, SEQ ID NO. 43, SEQ ID NO 44, SEQ ID NO. 45, SEQ ID NO. 46, SEQ ID NO. 47 and SEQ ID NO. 48.

13. The antibody or fragment according to claim 8 wherein the CDR3_{H} includes a peptide having a sequence defined by the following formula:
AA_{25H} AA_{26H} AA_{27H} AA_{28H} AA_{29H} AA_{30H} AA_{31H} AA_{32H} AA_{33H} AA_{34H} AA_{35H} AA_{36H} AA_{37H}
wherein:
AA_{25H}, is G, A, S or absent; AA_{26H}, is R, L, I, K or absent; AA_{27H}, A, N, V, F, Y, G or absent; AA_{28H}, is I, T, E, H, Y, L or absent, AA_{29H}, is Y, F, G or H; AA_{30H}, is Y, F, T, G, V or absent, AA_{31H}, is Y, A, S, N, L or absent, AA_{32H}, is K, M, T, Y or absent; AA_{33H}, is S, D or absent; AA_{34H}, is G, Y, A or absent; AA_{35H}, is F, M or absent; AA_{36H}, is A, V, D or S; AA_{37H}, is Y, F or S.

14. The antibody or fragment according to claim 13 wherein the peptide forming the CDR3_{H} is selected from the group consisting of: SEQ ID NO. 49, SEQ ID NO. 50, SEQ ID NO. 51, SEQ ID NO. 52, SEQ ID NO. 53, SEQ ID NO. 54, SEQ ID NO. 55 SEQ ID NO. 56 and SEQ ID NO. 57.


SEQUENCE LISTING (57 sequences)

SEQ ID NO: 1 - protein, 15 aa, Mus musculus
RASKSVSTSGYSYMS

SEQ ID NO: 2 - protein, 15 aa, Mus musculus
RASKSVSTSGYSYMS

SEQ ID NO: 3 - protein, 15 aa, Mus musculus
RASKSVSTSAYSYMH

SEQ ID NO: 4 - protein, 16 aa, Mus musculus
RSSQSLVHINGDTFLH

SEQ ID NO: 5 - protein, 16 aa, Mus musculus
RSSQSLVHINGDTFLQ

SEQ ID NO: 6 - protein, 16 aa, Mus musculus
RSSQNIVHSNGNTYLE

SEQ ID NO: 7 - protein, 16 aa, Mus musculus
RSSKSLLHSNGNTYLY

SEQ ID NO: 8 - protein, 14 aa, Mus musculus
RSSTGAVTTSNYAN

SEQ ID NO: 9 - protein, 14 aa, Mus musculus
RSSTGAVTTSNYAN

SEQ ID NO: 10 - protein, 11 aa, Mus musculus
RVSENIYSYLA

SEQ ID NO: 11 - protein, 7 aa, Mus musculus
LASNLES

SEQ ID NO: 12 - protein, 7 aa, Mus musculus
LASNLES

SEQ ID NO: 13 - protein, 7 aa, Mus musculus
LASNLES

SEQ ID NO: 14 - protein, 7 aa, Mus musculus
KVSNRFS

SEQ ID NO: 15 - protein, 7 aa, Mus musculus
KVSNRFS

SEQ ID NO: 16 - protein, 7 aa, Mus musculus
KVSYRFS

SEQ ID NO: 17 - protein, 7 aa, Mus musculus
RMSNLAS

SEQ ID NO: 18 - protein, 7 aa, Mus musculus
GTNNRAP

SEQ ID NO: 19 - protein, 7 aa, Mus musculus
DTNNRAP

SEQ ID NO: 20 - protein, 7 aa, Mus musculus
NAETLAE

SEQ ID NO: 21 - protein, 9 aa, Mus musculus
QHSRELPRT

SEQ ID NO: 22 - protein, 9 aa, Mus musculus
QHSRELPRT

SEQ ID NO: 23 - protein, 9 aa, Mus musculus
QHSRDLPRT

SEQ ID NO: 24 - protein, 9 aa, Mus musculus
SQITFVPWT

SEQ ID NO: 25 - protein, 9 aa, Mus musculus
SQITFVPWT

SEQ ID NO: 26 - protein, 9 aa, Mus musculus
FQGSLVPWT

SEQ ID NO: 27 - protein, 9 aa, Mus musculus
MQHLEYPLT

SEQ ID NO: 28 - protein, 9 aa, Mus musculus
GLWYSNHWV

SEQ ID NO: 29 - protein, 9 aa, Mus musculus
ALRSSNHLV

SEQ ID NO: 30 - protein, 9 aa, Mus musculus
QHHNVSPPT

SEQ ID NO: 31 - protein, 5 aa, Mus musculus
SGYWN

SEQ ID NO: 32 - protein, 5 aa, Mus musculus
SGYWN

SEQ ID NO: 33 - protein, 5 aa, Mus musculus
SGYWN

SEQ ID NO: 34 - protein, 5 aa, Mus musculus
EYALH

SEQ ID NO: 35 - protein, 5 aa, Mus musculus
GYAMS

SEQ ID NO: 36 - protein, 5 aa, Mus musculus
SYAMS

SEQ ID NO: 37 - protein, 5 aa, Mus musculus
SYWMS

SEQ ID NO: 38 - protein, 5 aa, Mus musculus
YYGMS

SEQ ID NO: 39 - protein, 5 aa, Mus musculus
NHGMN

SEQ ID NO: 40 - protein, 16 aa, Mus musculus
YINYSGNTYYNPSLNS

SEQ ID NO: 41 - protein, 16 aa, Mus musculus
YINYSGSTYYNPSLNS

SEQ ID NO: 42 - protein, 16 aa, Mus musculus
YINYSGTTYYNPSLKS

SEQ ID NO: 43 - protein, 16 aa, Mus musculus
GINPNNGGATYNQKFK

SEQ ID NO: 44 - protein, 17 aa, Mus musculus
TIDSGGYNTYYPDSVKG

SEQ ID NO: 45 - protein, 16 aa, Mus musculus
SIGYSGRTYYPDSVKG

SEQ ID NO: 46 - protein, 19 aa, Mus musculus
EIRLKSDNYATHYAESVKG

SEQ ID NO: 47 - protein, 17 aa, Mus musculus
WINTYTGEPTYADDFKG

SEQ ID NO: 48 - protein, 17 aa, Mus musculus
WINTYTGQSIYSDDFKG

SEQ ID NO: 49 - protein, 13 aa, Mus musculus
GRAIYYYKSGFAY

SEQ ID NO: 50 - protein, 13 aa, Mus musculus
GRAIFYYKSGFVY

SEQ ID NO: 51 - protein, 10 aa, Mus musculus
GRNTYSGFAF

SEQ ID NO: 52 - protein, 7 aa, Mus musculus
AYYAMDY

SEQ ID NO: 53 - protein, 8 aa, Mus musculus
SLVEFFDY

SEQ ID NO: 54 - protein, 13 aa, Mus musculus
GIFHYTSTSYFDY

SEQ ID NO: 55 - protein, 12 aa, Mus musculus
LVYYGNYDAMDY

SEQ ID NO: 56 - protein, 9 aa, Mus musculus
SRYLGVLSY

SEQ ID NO: 57 - protein, 7 aa, Mus musculus
GKGHFDS